SEQUENCE LISTING (4 sequences)

SEQ ID NO: 1 - DNA, 20 bp, Artificial Sequence
  source: /note=“Description of Artificial Sequence Synthetic oligonucleotide”
caggatacatttctacagct

SEQ ID NO: 2 - protein, 154 aa, Homo sapiens [sequence omitted]

SEQ ID NO: 3 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 4 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
caggatacatttctacagcu

(19) United States
(12) Patent Application Publication
(10) Pub. No.: US 2022/0073930 A1
(43) Pub. Date: Mar. 10, 2022

(54) COMPOSITIONS AND METHODS FOR TREATING AND PREVENTING AMYOTROPHIC LATERAL SCLEROSIS

(71) Applicants: Biogen MA Inc., Cambridge, MA (US); Ionis Pharmaceuticals, Inc., Carlsbad, CA (US)

(72) Inventors: Ivan Alexandrov Nestorov, Acton, MA (US); Toby Ferguson, Concord, MA (US); Daniel A. Norris, San Marcos, CA (US)

(21) Appl. No.: 17/291,199

(22) PCT Filed: Dec. 12, 2019
(86) PCT No.: PCT/US2019/065936

§ 371 Date: May 4, 2021

Related U.S. Application Data
(60) Provisional application No. 62/779,916, filed on Dec. 14, 2018.
(60) Provisional application No. 62/807,603, filed on Feb. 19, 2019.
(60) Provisional application No. 62/840,879, filed on Apr. 30, 2019.

(51) Int. Cl. C12N 15/113

(52) U.S. Cl. CPC C12N 15/1137; C12N 2310/11; C12N 2310/315; C12Y 115/01001; C12N 2310/3341; C12N 2310/3525; C12N 2320/35; C12N 2310/321

(57) ABSTRACT

Dosage regimens for SOD1-targeting antisense oligonucleotides, and salts thereof, are provided. These dosage regimens find use in the treatment of subjects having or at risk of developing amyotrophic lateral sclerosis.

### CROSS-REFERENCE TO RELATED APPLICATIONS

This applications claims the benefit of priority of U.S. Provisional Appl. Nos. 62/779,916, filed Dec. 14, 2018; 62/807,603, filed Feb. 19, 2019; and 62/840,879 filed Apr. 30, 2019, the contents of all of which are incorporated by reference herein in their entirety.

### FIELD

The present application relates generally to dosage regimens for the clinical use of antisense oligonucleotides, or salts thereof, that reduce expression of superoxide dismutase 1 (SOD1) in a human subject in need thereof, e.g., adults with amyotrophic lateral sclerosis (ALS) who have a confirmed mutation of the human SOD1 gene. Such methods are useful to treat, prevent, or ameliorate ALS by inhibiting expression of SOD1.

### BACKGROUND

The soluble SOD1 enzyme (also known as Cu/Zn superoxide dismutase) is one of the superoxide dismutases that provides defense against oxidative damage of biomolecules by catalyzing the dismutation of superoxide to hydrogen peroxide (H₂O₂) (Fridovich, Annu. Rev. Biochem., 64:97-112 (1995)). The superoxide anion (O²⁻) is a potentially harmful cellular by-product produced primarily by errors of oxidative phosphorylation in mitochondria (Turrens, J. Physiol., 552:335-344 (2003))

Amyotrophic Lateral Sclerosis (ALS, also known as Lou Gehrig's disease) is a devastating progressive neurodegenerative disease affecting as many as 30,000 Americans at any given time. Mutations in the SOD1 gene are associated with a dominantly-inherited form of ALS, a disorder characterized by a selective degeneration of upper and lower motor neurons (Rowland, N. Engl. J. Med., 2001, 344:1688-1700 (2001)). There is a tight genetic linkage between familial ALS and missense mutations in the SOD1 gene (Rosen, Nature, 362:59-62 (1993)).

The toxicity of mutant SOD1 is believed to arise from an initial misfolding (gain of function) reducing nuclear protection from the active enzyme (loss of function in the nuclei), a process that may be involved in ALS pathogenesis (Sau, Hum. Mol. Genet., 16:1604-1618 (2007)). The progressive degeneration of the motor neurons in ALS eventually leads to their death. When the motor neurons die, the ability of the brain to initiate and control muscle movement is lost. With voluntary muscle action progressively affected, patients in the later stages of the disease may become totally paralyzed.

Currently lacking are acceptable options for treating such neurodegenerative diseases. It is therefore an object herein to provide methods for the treatment of such diseases.

### SUMMARY

This disclosure relates, in part, to dosage regimens of antisense oligonucleotides that reduce expression of superoxide dismutase 1 (SOD1) and the use of such antisense oligonucleotides, or salts thereof, to inhibit expression of SOD1 and to treat, prevent, or ameliorate ALS in a human subject with a mutation in the SOD1 gene.

In a first aspect, the disclosure features a method of treating or preventing amyotrophic lateral sclerosis associated with a mutation in the human superoxide dismutase 1 (SOD1) gene in a human subject in need thereof. The method involves administering to the human subject (e.g., by intrathecal administration) a pharmaceutical composition in an amount sufficient to deliver a fixed dose of about 100 mg or 100 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In a second aspect, the disclosure features a method of treating or preventing amyotrophic lateral sclerosis associated with a mutation in the human SOD1 gene in a human subject in need thereof. The method involves administering to the human subject (e.g., by intrathecal administration) a pharmaceutical composition in an amount sufficient to deliver a fixed dose of about 60 mg or 60 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In a third aspect, the disclosure features a method of treating or preventing amyotrophic lateral sclerosis associated with a mutation in the human SOD1 gene in a human subject in need thereof. The method involves administering to the human subject (e.g., by intrathecal administration) a pharmaceutical composition in an amount sufficient to deliver a fixed dose of about 40 mg or 40 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In a fourth aspect, the disclosure features a method of treating or preventing amyotrophic lateral sclerosis associated with a mutation in the human SOD1 gene in a human subject in need thereof. The method involves administering to the human subject (e.g., by intrathecal administration) a pharmaceutical composition in an amount sufficient to deliver a fixed dose of about 20 mg or 20 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In a fifth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition in an amount sufficient to deliver a fixed dose of about 100 mg or 100 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In a sixth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition in an amount sufficient to deliver a fixed dose of about 60 mg or 60 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In a seventh aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition in an amount sufficient to deliver a fixed dose of about 40 mg or 40 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In an eighth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition in an amount sufficient to deliver a fixed dose of about 20 mg or 20 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

In some embodiments of the above aspects, the mutation in the SOD1 gene is A4V, H46R, G93S, A4T, G141X, D133A, V148G, N139K, G85R, G93A, V14G, C6S, I113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, or G147R. In one embodiment, the mutation in the SOD1 gene is A4V. In another embodiment, the mutation in the SOD1 gene is H46R. In yet another embodiment, the mutation in the SOD1 gene is G93S.

In some embodiments, the mutation in the human SOD1 gene is identified by a genetic test.

In certain embodiments, the methods above further involve identifying the mutation in the human SOD1 gene by a genetic test.

In some embodiments, the pharmaceutical composition is administered to the human subject at least 5 times (e.g., 5, 6, 7, 8, 9, 10, 11, 12, 13, 14, 15, 16, 17, 18, 19, 20, 21, 22, 23, 24 times) over the course of four months.

In certain embodiments, the human subject is administered a loading dose or loading doses of the pharmaceutical composition followed by a maintenance dose or maintenance doses. In some instances, three loading doses are administered, wherein the second loading dose is administered about two weeks after or two weeks after the first loading dose, and the third loading dose is administered about two weeks after or two weeks after the second loading dose (e.g., the loading doses are administered on day 1, day 15, and day 29). In some instances, the maintenance doses are administered about every 4 weeks or 4 weeks beginning 4 weeks after the third loading dose (e.g., for 1 month, 2 months, three months, four months, five months, six months, seven months, eight months, nine months, ten months).

In certain embodiments, the human subject is administered three loading doses of the pharmaceutical composition followed by at least one (e.g., 1, 2, 3, 4, 5, 6, 7, 8, 9, 10, 11, 12) maintenance dose. In some instances, the three loading doses are administered about two weeks or two weeks apart. In some instances, the three loading doses are administered about 14 days or 14 days apart. In some instances, the maintenance dose/doses are administered beginning about 4 weeks or 4 weeks after the third loading dose. In some instances, the maintenance dose/doses are administered every month beginning one month after the third loading dose. In some instances, the maintenance dose/doses are administered every 28 days beginning 28 days after the third loading dose.

In certain embodiments, loading doses and maintenance doses of the pharmaceutical composition are administered to the human subject as follows:

(i) a first loading dose of the pharmaceutical composition;

(ii) a second loading dose of the pharmaceutical composition administered 14 days after the first loading dose;

(iii) a third loading dose of the pharmaceutical composition administered 28 days after the first loading dose; and

(iv) a first maintenance dose of the pharmaceutical composition administered 28 days or 1 month after the third loading dose.

In certain embodiments, loading doses and maintenance doses of the pharmaceutical composition are administered to the human subject as follows:

(i) a first loading dose in an amount sufficient to deliver a fixed dose of 100 mg of the antisense oligonucleotide;

(ii) a second loading dose in an amount sufficient to deliver a fixed dose of 100 mg of the antisense oligonucleotide, wherein the second loading dose is administered 14 days after the first loading dose;

(iii) a third loading dose in an amount sufficient to deliver a fixed dose of 100 mg of the antisense oligonucleotide, wherein the third loading dose is administered 28 days after the first loading dose; and

(iv) a first maintenance dose in an amount sufficient to deliver a fixed dose of 100 mg of the antisense oligonucleotide, wherein the first maintenance dose is administered 28 days after the third loading dose.

In certain embodiments, loading doses and maintenance doses of the pharmaceutical composition are administered to the human subject as follows:

(i) a first loading dose in an amount sufficient to deliver a fixed dose of 100 mg of the antisense oligonucleotide;

(ii) a second loading dose in an amount sufficient to deliver a fixed dose of 100 mg of the antisense oligonucleotide, wherein the second loading dose is administered 14 days after the first loading dose;

(iii) a third loading dose in an amount sufficient to deliver a fixed dose of 100 mg of the antisense oligonucleotide, wherein the third loading dose is administered 28 days after the first loading dose; and

(iv) a first maintenance dose in an amount sufficient to deliver a fixed dose of 100 mg of the antisense oligonucleotide, wherein the first maintenance dose is administered 1 month after the third loading dose.

In a ninth aspect, the disclosure provides a method of treating or preventing amyotrophic lateral sclerosis associated with a mutation in the SOD1 gene in a human subject in need thereof. The method involves administering to the human subject by intrathecal administration a pharmaceutical composition comprising an antisense oligonucleotide or a salt thereof, wherein the antisense oligonucleotide has the following structure:

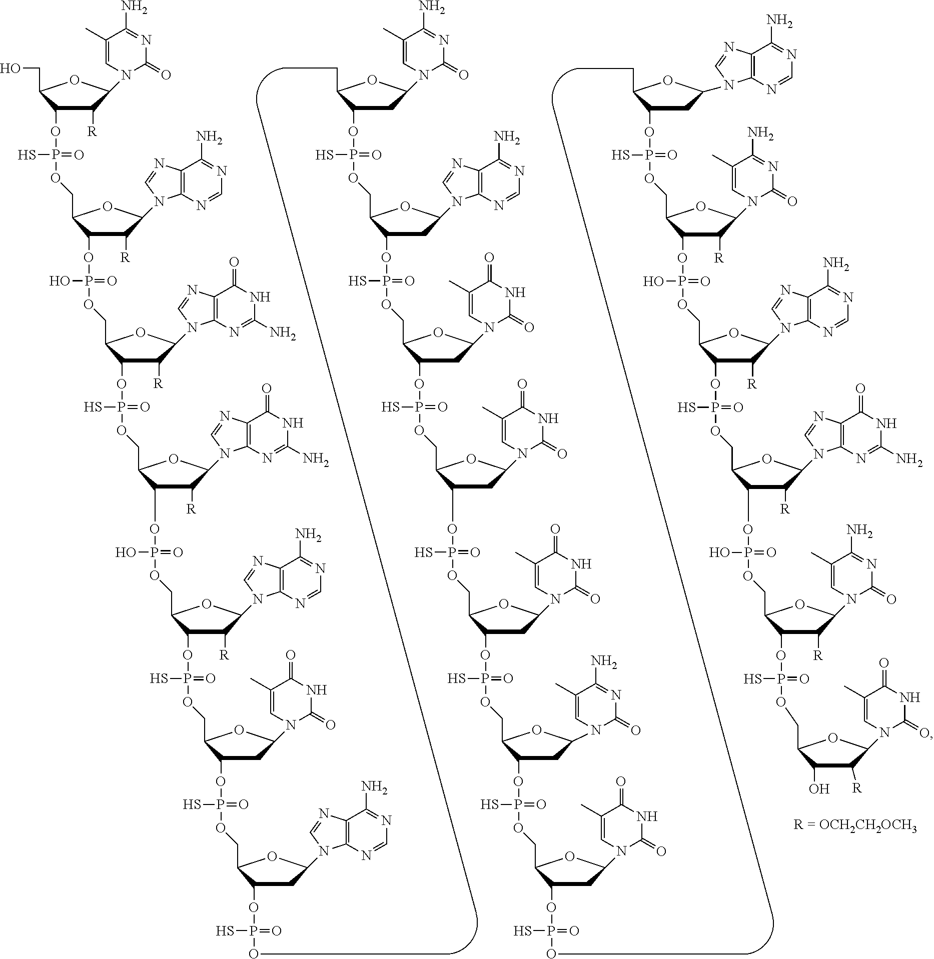

and wherein the antisense oligonucleotide or the salt thereof is administered at a dose equivalent to 100 mg of the antisense oligonucleotide. 105.9 mg of Compound A (i.e., the nonadecasodium salt of ISIS 666853) is equivalent to 100 mg of the antisense oligonucleotide. The pharmaceutical composition administered may comprise the antisense oligonucleotide, one or more salts of the antisense oligonucleotide, or mixtures thereof.

In a tenth aspect, the disclosure provides a method of treating or preventing amyotrophic lateral sclerosis associated with a mutation in the SOD1 gene in a human subject in need thereof. The method involves administering to the human subject by intrathecal administration a pharmaceutical composition comprising an antisense oligonucleotide or a salt thereof, wherein the antisense oligonucleotide has the following structure:

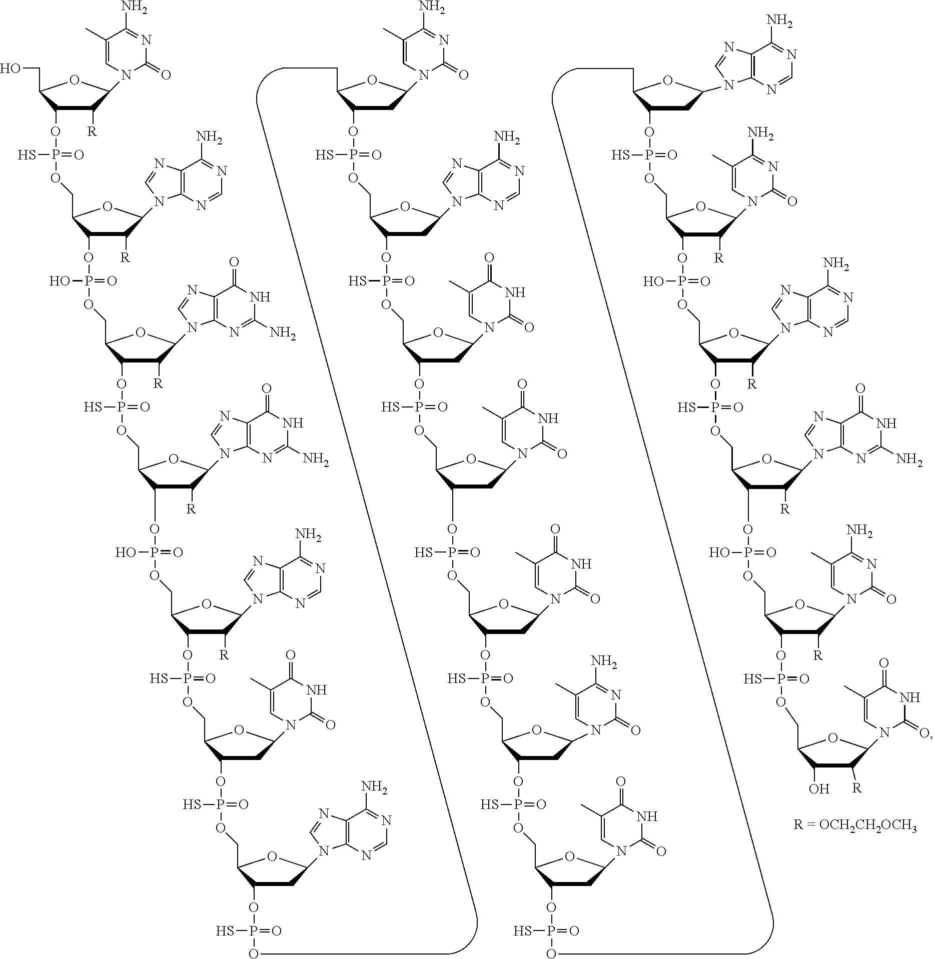

and wherein the antisense oligonucleotide or the salt thereof is administered at a dose equivalent to 60 mg of the antisense oligonucleotide. 63.5 mg of Compound A is equivalent to 60 mg of the antisense oligonucleotide. The pharmaceutical composition administered may comprise the antisense oligonucleotide, one or more salts of the antisense oligonucleotide, or mixtures thereof.

In an eleventh aspect, the disclosure provides a method of treating or preventing amyotrophic lateral sclerosis associated with a mutation in the SOD1 gene in a human subject in need thereof. The method involves administering to the human subject by intrathecal administration a pharmaceutical composition comprising an antisense oligonucleotide or a salt thereof, wherein the antisense oligonucleotide has the following structure:

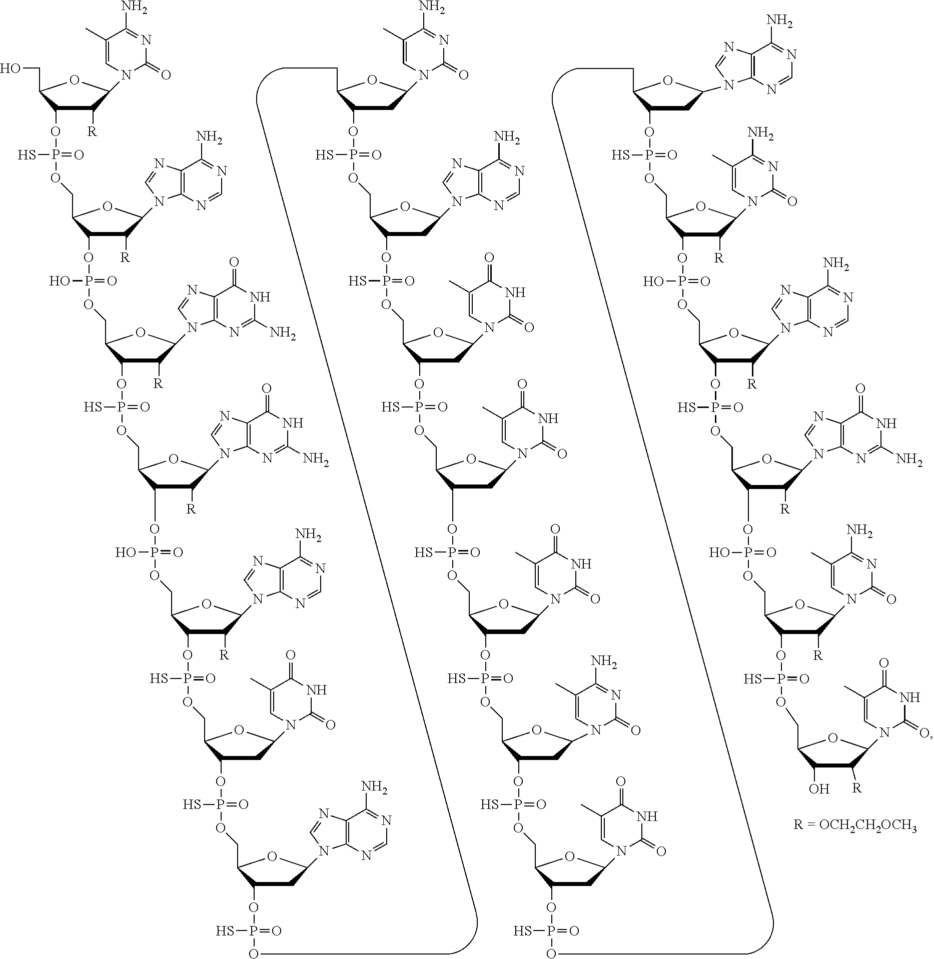

and wherein the antisense oligonucleotide or the salt thereof is administered at a dose equivalent to 40 mg of the antisense oligonucleotide. 42.3 mg of Compound A is equivalent to 40 mg of the antisense oligonucleotide. The pharmaceutical composition administered may comprise the antisense oligonucleotide, one or more salts of the antisense oligonucleotide, or mixtures thereof.

In a twelfth aspect, the disclosure provides a method of treating or preventing amyotrophic lateral sclerosis associated with a mutation in the SOD1 gene in a human subject in need thereof. The method involves administering to the human subject by intrathecal administration a pharmaceutical composition comprising an antisense oligonucleotide or a salt thereof, wherein the antisense oligonucleotide has the following structure:

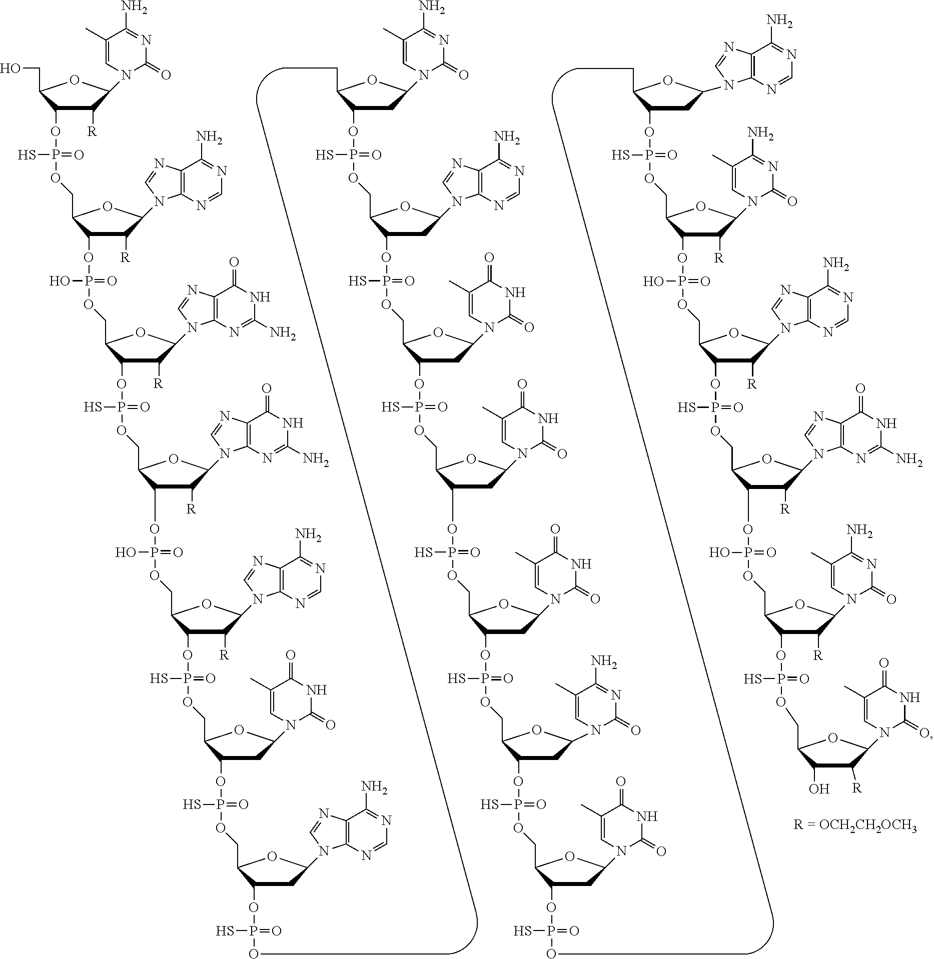

and wherein the antisense oligonucleotide or the salt thereof is administered at a dose equivalent to 20 mg of the antisense oligonucleotide. 21.2 mg of Compound A is equivalent to 20 mg of the antisense oligonucleotide. The pharmaceutical composition administered may comprise the antisense oligonucleotide, one or more salts of the antisense oligonucleotide, or mixtures thereof.

In a thirteenth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition comprising an antisense oligonucleotide or a salt thereof, wherein the antisense oligonucleotide has the following structure:

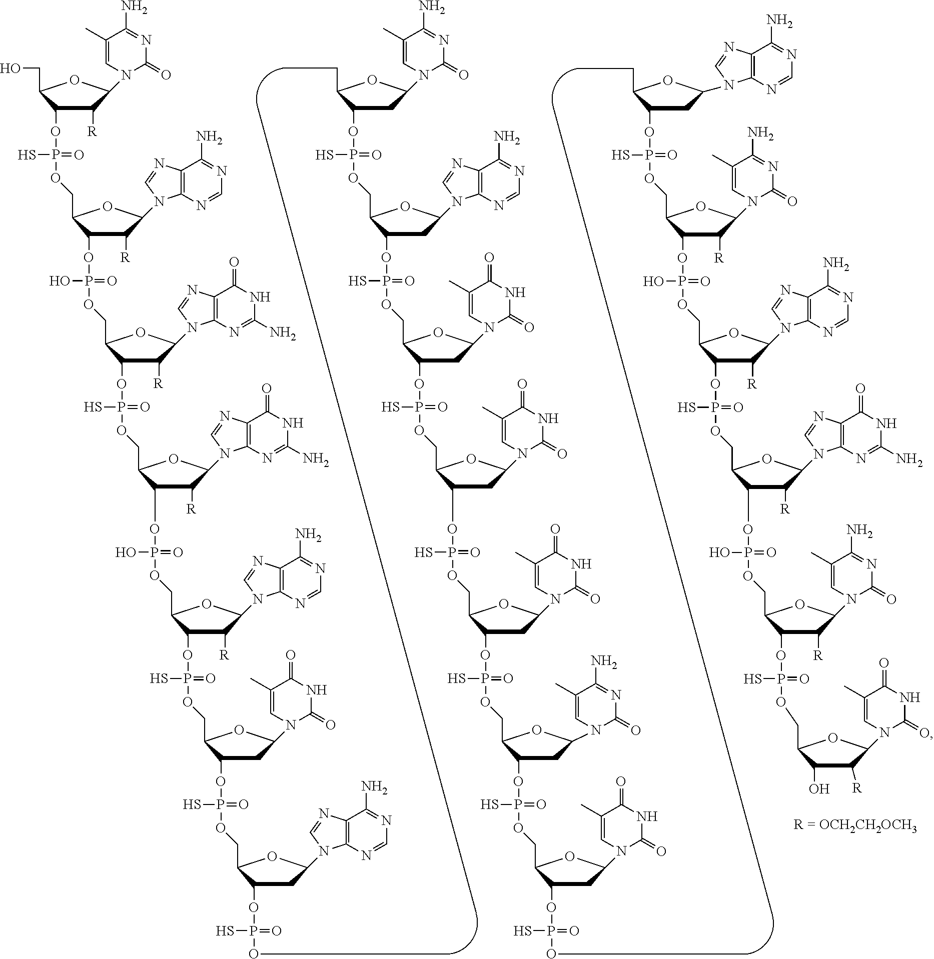

and wherein the antisense oligonucleotide or the salt thereof is administered at a dose equivalent to 100 mg of the antisense oligonucleotide. 105.9 mg of Compound A is equivalent to 100 mg of the antisense oligonucleotide. The pharmaceutical composition administered may comprise the antisense oligonucleotide, one or more salts of the antisense oligonucleotide, or mixtures thereof.

In a fourteenth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition comprising an antisense oligonucleotide or a salt thereof, wherein the antisense oligonucleotide has the following structure:

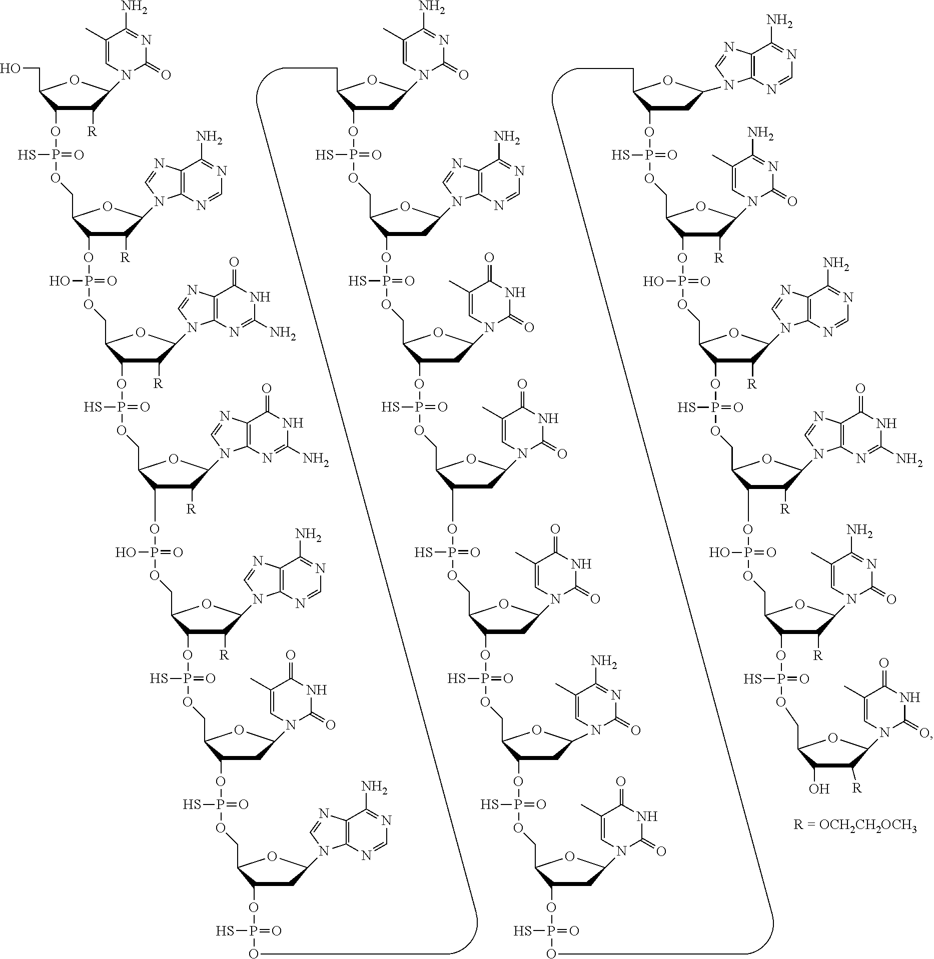

and wherein the antisense oligonucleotide or the salt thereof is administered at a dose equivalent to 60 mg of the antisense oligonucleotide. 63.5 mg of Compound A is equivalent to 60 mg of the antisense oligonucleotide. The pharmaceutical composition administered may comprise the antisense oligonucleotide, one or more salts of the antisense oligonucleotide, or mixtures thereof.

In a fifteenth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition comprising an antisense oligonucleotide or a salt thereof, wherein the antisense oligonucleotide has the following structure:

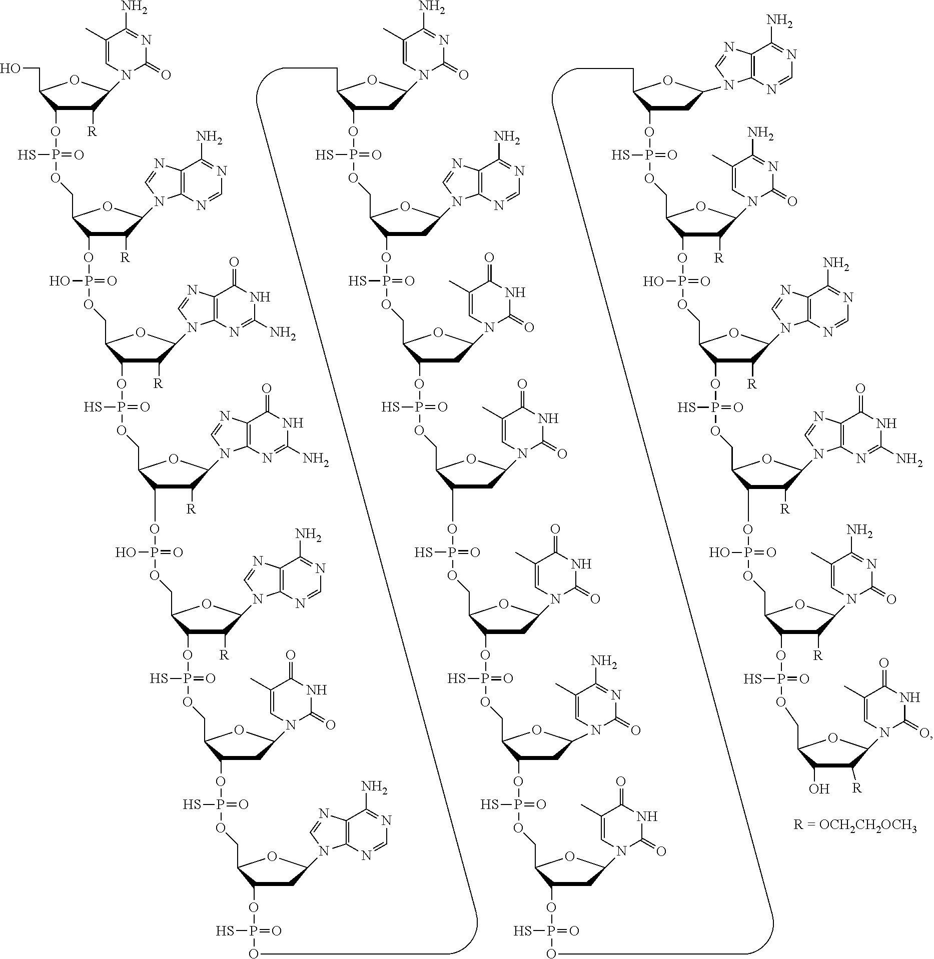

and wherein the antisense oligonucleotide or the salt thereof is administered at a dose equivalent to 40 mg of the antisense oligonucleotide. 42.3 mg of Compound A is equivalent to 40 mg of the antisense oligonucleotide. The pharmaceutical composition administered may comprise the antisense oligonucleotide, one or more salts of the antisense oligonucleotide, or mixtures thereof.

In a sixteenth aspect, the disclosure provides a method of reducing human SOD1 protein synthesis or human SOD1 mRNA levels in a human subject having a mutation in the human SOD1 gene associated with amyotrophic lateral sclerosis. The method involves administering to the human subject by intrathecal administration a pharmaceutical composition comprising an antisense oligonucleotide or a salt thereof, wherein the antisense oligonucleotide has the following structure:

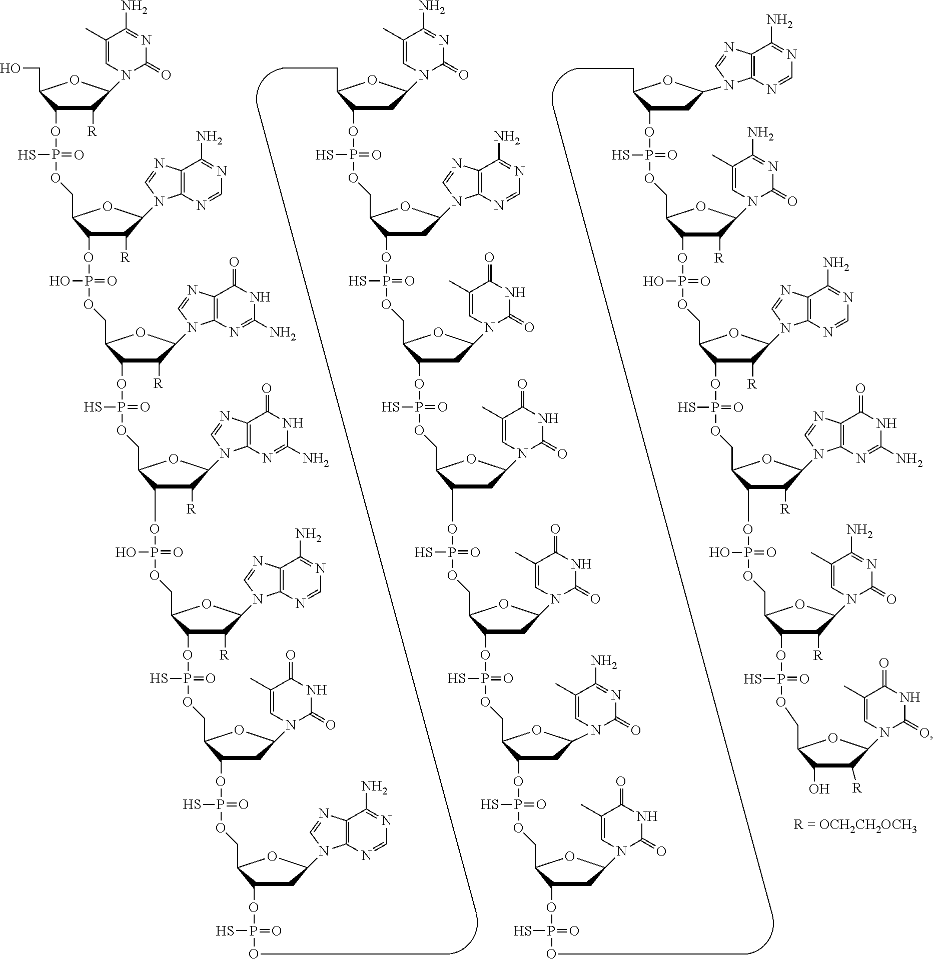

and wherein the antisense oligonucleotide or the salt thereof is administered at a dose equivalent to 20 mg of the antisense oligonucleotide. 21.2 mg of Compound A is equivalent to 20 mg of the antisense oligonucleotide. The pharmaceutical composition administered may comprise the antisense oligonucleotide, one or more salts of the antisense oligonucleotide, or mixtures thereof.

In some embodiments, the human subject is administered a salt of the antisense oligonucleotide. In some embodiments, the salt is a sodium salt. In some embodiments, the salt of the antisense oligonucleotide has the following structure:

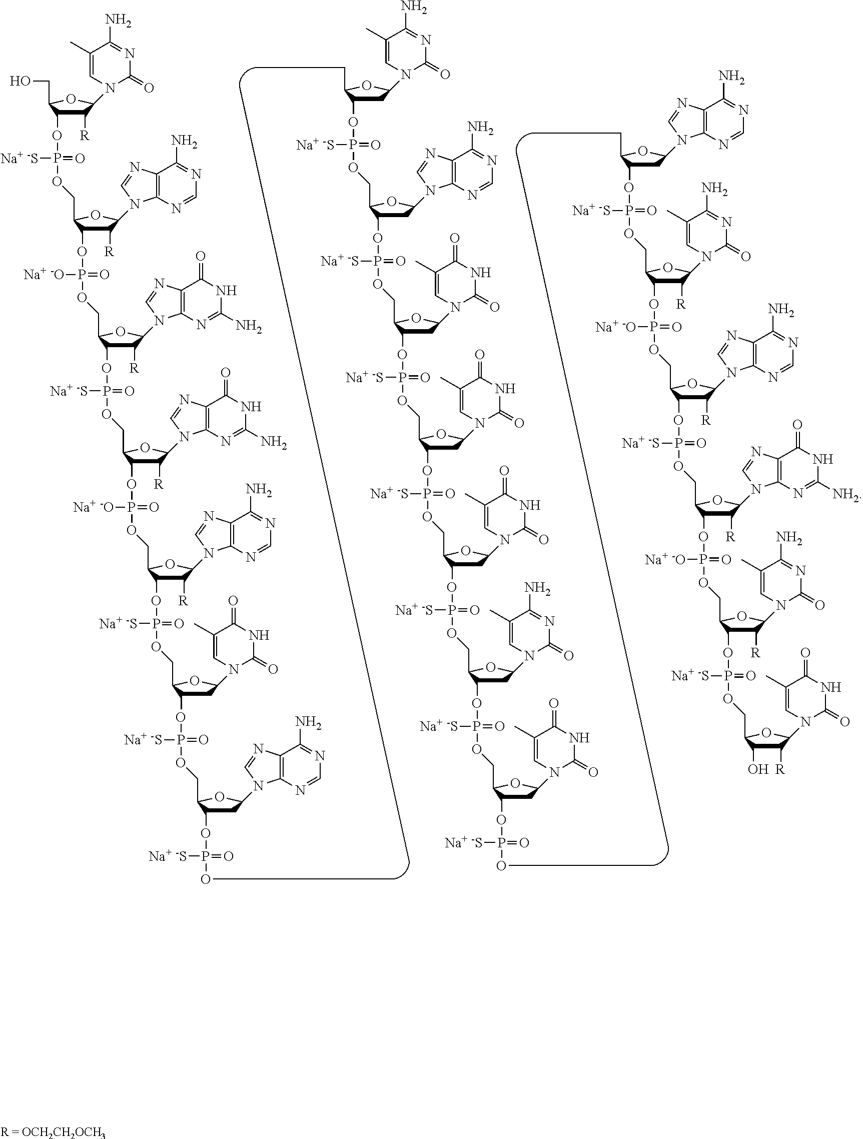

In some embodiments of the above aspects, the mutation in the SOD1 gene is A4V, H46R, G93S, A4T, G141X, D133A, V148G, N139K, G85R, G93A, V14G, C6S, I113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, or G147R. In one embodiment, the mutation in the SOD1 gene is A4V. In another embodiment, the mutation in the SOD1 gene is H46R. In yet another embodiment, the mutation in the SOD1 gene is G93S.

In some embodiments, the mutation in the human SOD1 gene is identified by a genetic test.

In certain embodiments, the methods above further involve identifying the mutation in the human SOD1 gene by a genetic test.

In some embodiments, the pharmaceutical composition is administered to the human subject at least 5 times over the course of four months.

In certain embodiments, the human subject is administered a loading dose or loading doses of the pharmaceutical composition followed by a maintenance dose or maintenance doses. In some instances, three loading doses are administered, wherein the loading doses are separated by two weeks for e.g., on day 1, day 15, and day 29. In some instances, the maintenance doses are administered every 4 weeks beginning 4 weeks after the third loading dose (e.g., for 1 month, 2 months, three months, four months, five months, six months, seven months, eight months, nine months, ten months).

In certain embodiments, the human subject is administered three loading doses of the pharmaceutical composition followed by at least one (e.g., 1, 2, 3, 4, 5, 6, 7, 8, 9, 10, 11, 12) maintenance dose. In some instances, the three loading doses are administered two weeks apart. In some instances, the three loading doses are administered 14 days apart. In some instances, the maintenance dose/doses are administered every 4 weeks beginning 4 weeks after the third loading dose. In some instances, the maintenance dose/doses are administered every month beginning one month after the third loading dose. In some instances, the maintenance dose/doses are administered every 28 days beginning 28 days after the third loading dose.

In certain embodiments, loading doses and maintenance doses of the pharmaceutical composition are administered to the human subject as follows:

(i) a first loading dose of the pharmaceutical composition;

(ii) a second loading dose of the pharmaceutical composition administered 14 days after the first loading dose;

(iii) a third loading dose of the pharmaceutical composition administered 28 days after the first loading dose; and

(iv) a first maintenance dose of the pharmaceutical composition administered 28 days or 1 month after the third loading dose.

In certain embodiments, loading doses and maintenance doses of the pharmaceutical composition are administered to the human subject as follows:

(i) a first loading dose equivalent to 100 mg of the antisense oligonucleotide;

(ii) a second loading dose equivalent to 100 mg of the antisense oligonucleotide, wherein the second loading dose is administered 14 days after the first loading dose;

(iii) a third loading dose equivalent to 100 mg of the antisense oligonucleotide, wherein the third loading dose is administered 28 days after the first loading dose; and

(iv) a first maintenance dose equivalent to 100 mg of the antisense oligonucleotide, wherein the first maintenance dose is administered 28 days after the third loading dose.

In certain embodiments, loading doses and maintenance doses of the pharmaceutical composition are administered to the human subject as follows:

(i) a first loading dose equivalent to 100 mg of the antisense oligonucleotide;

(ii) a second loading dose equivalent to 100 mg of the antisense oligonucleotide, wherein the second loading dose is administered 14 days after the first loading dose;

(iii) a third loading dose equivalent to 100 mg of the antisense oligonucleotide, wherein the third loading dose is administered 28 days after the first loading dose; and

(iv) a first maintenance dose equivalent to 100 mg of the antisense oligonucleotide, wherein the first maintenance dose is administered 1 month after the third loading dose.

In another aspect, the disclosure features a syringe or pump comprising a sterile preparation of an antisense oligonucleotide. The syringe or pump is adapted for intrathecal administration of the antisense oligonucleotide at a fixed dose of 20 mg, 40 mg, 60 mg, or 100 mg. The nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF DRAWINGS

FIG. 1 is a graphical depiction of mean disease duration (years from symptom onset) for patients with different human SOD1 mutations.

FIG. 2 depicts the dose-dependent increases in Compound A cerebrospinal fluid (CSF) concentrations observed in multiple ascending dose (MAD) cohorts. The top broken line represents the 100 mg dose; the next two broken lines the 60 and 40 mg doses, and the bottom most broken line represents the 20 mg dose.

FIG. 3 depicts the dose-dependent decreases in CSF SOD1 concentrations observed in MAD cohorts. At study day 85, the top circle corresponds to 20 mg; the next circle below to placebo; the next circle below to 60 mg; the next circle below to 40 mg; and the bottom most circle to 100 mg of Compound A.

FIG. 4A is a plot of LS mean change from baseline in Amyotrophic Lateral Sclerosis Functional Rating Scale-Revised (ALFSFRS-R) in patients in MAD cohorts. At study day 85, the top circle corresponds to 40 mg of Compound A; the next circle below to 100 mg of Compound A; the next circle below to 60 mg of Compound A; the next circle below to 20 mg of Compound A; and the bottom most circle to placebo.

FIG. 4B is a plot of LS mean change from baseline (90% CI) in percent predicted Slow Vital Capacity (SVC) in patients in MAD cohorts. At study day 85, the top circle corresponds to 60 mg of Compound A; the next circle below to 40 mg of Compound A; the next circle below to 100 mg of Compound A; the next circle below to 20 mg of Compound A; and the bottom most circle to placebo.

FIG. 4C is a plot of LS mean change from baseline (90% CI) in hand-held dynamometry (HHD) overall megascore in patients in MAD cohorts. At study day 92, the top circle corresponds to 40 mg of Compound A; the next circle below to 100 mg of Compound A; the next circle below to 60 mg of Compound A; the next circle below to 20 mg of Compound A; and the bottom most circle to placebo.

FIG. 5 illustrates changes in ALSFRS-R, SVC, HHD megascore and CSF SOD1 level from baseline to Day 85 in patients with fast-progressing mutations receiving either placebo or 100 mg of Compound A.

FIG. 6 illustrates changes in ALSFRS-R, SVC, HHD megascore and CSF SOD1 level from baseline to Day 85 in patients with slow-progressing mutations receiving either placebo or 100 mg of Compound A.

FIG. 7 depicts the effect of treatment with 100 mg of Compound A on pNFH levels from baseline to Day 85 in patients with fast-progressing SOD1 mutations.

FIG. 8 depicts the effect of treatment with 100 mg of Compound A on pNFH levels from baseline to Day 85 in patients with slow-progressing SOD1 mutations.

### DETAILED DESCRIPTION

This disclosure features dosage regimens of antisense oligonucleotides, or salts thereof, that reduce expression of superoxide dismutase 1 (SOD1) and the use of such antisense oligonucleotides, or salts thereof, to treat, prevent, or ameliorate amyotrophic lateral sclerosis (ALS) in adults having a mutation of the human SOD1 gene.

### Definitions

“2′-O-methoxyethyl” (also 2′-MOE and 2′-OCH₂CH₂—OCH₃ and MOE) refers to an O-methoxy-ethyl modification of the 2′ position of a furanose ring. A 2′-O-methoxyethyl modified sugar is a modified sugar.

“2′-MOE nucleoside” (also 2′-O-methoxyethyl nucleoside) means a nucleoside comprising a MOE modified sugar moiety.

“5-methylcytosine” means a cytosine modified with a methyl group attached to the 5′ position. A 5-methylcytosine is a modified nucleobase.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“About” in the context of the amount of a substance means +/−10% of the indicated value. So, about 100 mg of an antisense oligonucleotide includes 90 mg to 110 mg of the antisense oligonucleotide. In the context of temporal units, e.g., about 10 days or about 1 week, “about” means +/−3 days.

“Intrathecal or IT” means administration into the cerebrospinal fluid under the arachnoid membrane which covers the brain and spinal cord.

“Loading Dose” means a dose administered during a dosing phase during which administration is initiated and steady state concentration of the drug (e.g., antisense oligonucleotide) achieved.

“Maintenance Dose” means a dose administered during a dosing phase after steady state concentration of the drug (e.g., antisense oligonucleotide) has been achieved.

“Fixed dose” refers to a predetermined quantity of antisense oligonucleotide (e.g., 20 mg, 40 mg, 60 mg, 100 mg) intended to achieve a desired therapeutic concentration (e.g., steady state concentration) or effect in the subject.

### Amyotrophic Lateral Sclerosis

Amyotrophic lateral sclerosis (ALS) is a rare neurodegenerative disease resulting in loss of motor neurons within the cortex, brainstem, and spinal cord. Patients suffer from the progressive loss of muscle mass, strength, and function in bulbar, respiratory, and voluntary muscles. Decline is inevitable, with death, typically from respiratory failure, occurring 2 to 5 years, on average, following diagnosis. Although the majority of patients suffer from sporadic ALS, a smaller fraction of patients, approximately 2%, have an inherited, or familial, form of ALS caused by a variety of mutations in superoxide dismutase 1 (SOD1). Over 180 SOD1 mutations have been reported to cause this form of ALS (referred to as SOD1 ALS) since its initial discovery in 1993. The Amyotrophic Lateral Sclerosis Online Genetics Database (ALSoD). Institute of Psychiatry, Psychology & Neuroscience. Published 2015; Rosen, Nature, 364(6435):362 (1993)). Disease progression for individual mutations is variable, with survival of less than 15 months with the most severe mutations. Although the mechanism by which mutations cause SOD1 ALS is not known, compelling data suggest that toxic gain of function, not loss of SOD1 activity, is the trigger that initiates the cascade of events resulting in motor neuron death (Bruijn et al., Science, 281(5384):1851-4 (1998)).

Approved treatments for ALS are riluzole (Rilutek®) and edaravone (Radicava™) Riluzole provides a modest increase in survival (2 to 3 months) without demonstrable improvement in strength or disability. Edaravone lessens functional decline as measured by the Amyotrophic Lateral Sclerosis Functional Rating Scale-Revised (ALSFRS-R). The effect of edaravone on survival is unknown. No SOD1-specific ALS treatments are available.

### Superoxide Dismutase 1

Superoxide dismutase [Cu—Zn] also known as superoxide dismutase 1 (SOD1) is an enzyme that in humans is encoded by the SOD1 gene, located on chromosome 21.

SOD1 is a 32 kDa homodimer that forms a β-barrel and contains an intramolecular disulfide bond and a binuclear Cu/Zn site in each subunit. This Cu/Zn site holds the copper and a zinc ion and is responsible for catalyzing the disproportionation of superoxide to hydrogen peroxide and dioxygen.

SOD1 is one of three superoxide dismutases responsible for destroying free superoxide radicals in the body. The encoded isozyme is a soluble cytoplasmic and mitochondrial intermembrane space protein, acting as a homodimer to convert naturally occurring, but harmful, superoxide radicals to molecular oxygen and hydrogen peroxide. Hydrogen peroxide can then be broken down by another enzyme called catalase.

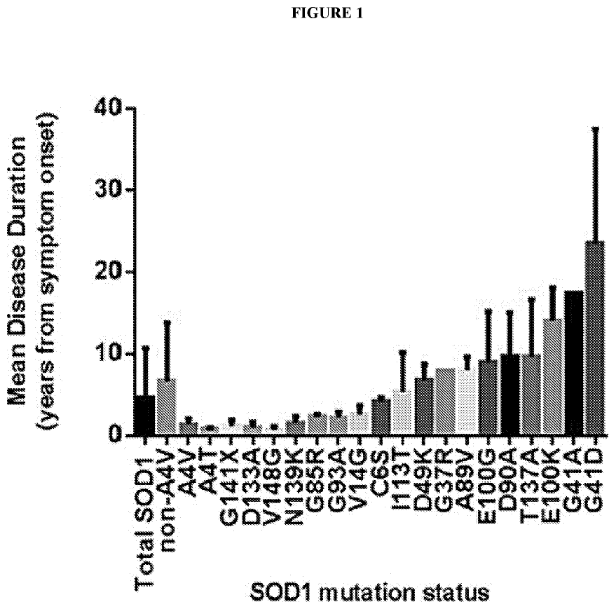

At least 180 mutations in the SOD1 gene have been linked to familial ALS (Conwit R A, J Neurol Sci., 251 (1-2):1-2 (2006); Al-Chalabi A, Leigh P N, Curr. Opin. in Neurol., 13(4):397-405 (2000); Redler R L, Dokholyan N V, Progress in Molecular Biology and Translational Science, 107:215-62 (2012)). However, wild-type SOD1, under conditions of cellular stress, has also been implicated in a significant fraction of sporadic ALS cases, which represent 90% of ALS patients. The most frequent mutations in human SOD1 are A4V in the United States; H46R in Japan; and G93S in Iceland. Other well-known human SOD1 mutations include: A4T, G141X, D133A, V148G, N139K, G85R, G93A, V14G, C6S, I113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, and G147R. There is significant heterogeneity in disease duration based on the SOD1 mutation (see, FIG. 1). Virtually all known ALS-causing SOD1 mutations act in a dominant fashion; a single mutant copy of the SOD1 gene is sufficient to cause the disease.

The amino acid sequence of human SOD1 can be found at UniProt P00441 and GENBANK Accession No. NP_000445, and is provided below:

(SEQ ID NO: 2)
MATKAVCVLK GDGPVQGIIN FEQKESNGPV KVWGSIKGLT
EGLHGFHVHE FGDNTAGCTS AGPHFNPLSR KHGGPKDEER
HVGDLGNVTA DKDGVADVSI EDSVISLSGD HCIIGRTLVV
HEKADDLGKG GNEESTKTGN AGSRLACGVI GIAQ

The nucleotide sequence encoding human SOD1 is provided at GENBANK Accession No. NM_000454.4, and is also provided below (the region recognized by the antisense oligonucleotide of this disclosure is underlined):

| (SEQ ID NO: 3) | |
|---|---|
| 1 | gtttggggcc agagtgggcg aggcgcggag gtctggccta taaagtagtc gcggagacgg |
| 61 | ggtgctggtt tgcgtcgtag tctcctgcag cgtctggggt ttccgttgca gtcctcggaa |
| 121 | ccaggacctc ggcgtggcct agcgagttat ggcgacgaag gccgtgtgcg tgctgaaggg |
| 181 | cgacggccca gtgcagggca tcatcaattt cgagcagaag gaaagtaatg gaccagtgaa |
| 241 | ggtgtgggga agcattaaag gactgactga aggcctgcat ggattccatg ttcatgagtt |
| 301 | tggagataat acagcaggct gtaccagtgc aggtcctcac tttaatcctc tatccagaaa |
| 361 | acacggtggg ccaaaggatg aagagaggca tgttggagac ttgggcaatg tgactgctga |
| 421 | caaagatggt gtggccgatg tgtctattga agattctgtg atctcactct caggagacca |
| 481 | ttgcatcatt ggccgcacac tggtggtcca tgaaaaagca gatgacttgg gcaaaggtgg |
| 541 | aaatgaagaa agtacaaaga caggaaacgc tggaagtcgt ttggcttgtg gtgtaattgg |
| 601 | gatcgcccaa taaacattcc cttggatgta gtctgaggcc ccttaactca tctgttatcc |
| 661 | tgctagctgt agaaatgtat cctgataaac attaaacact gtaatcttaa aagtgtaatt |
| 721 | gtgtgacttt ttcagagttg ctttaaagta cctgtagtga gaaactgatt tatgatcact |
| 781 | tggaagattt gtatagtttt ataaaactca gttaaaatgt ctgtttcaat gacctgtatt |
| 841 | ttgccagact taaatcacag atgggtatta aacttgtcag aatttctttg tcattcaagc |
| 901 | ctgtgaataa aaaccctgta tggcacttat tatgaggcta ttaaaagaat ccaaattcaa |
| 961 | actaaaaaaa aaaaaaaaaa a |

### ISIS 666853

ISIS 666853 is a 5-10-5 MOE gapmer, having the sequence of (from 5′ to 3′) CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. ISIS 666853 is described by the following chemical notation: mCes Aeo Ges Geo Aes Tds Ads mCds Ads Tds Tds Tds mCds Tds Ads mCeo Aes Geo mCes Te; wherein,

A=an adenine,

mC=a 5-methylcytosine

G=a guanine,

T=a thymine,

e=a 2′-O-methoxyethylribose modified sugar,

d=a 2′-deoxyribose sugar,

s=a phosphorothioate internucleoside linkage, and

o=a phosphodiester internucleoside linkage.

The ISIS 666853 sequence can also be written in shorthand as follows:

5′-^{Me}CA_{P═O}GG_{P═O}ATA^{Me}CATTT^{Me}CTA^{Me}C_{P═O}AG_{P═O}^{Me}C^{Me}U-3′

The underlined residues are 2′-MOE nucleosides. The P═O annotation reflects the location of phosphate diester linkages.

ISIS 666853 is depicted by the following chemical structure:

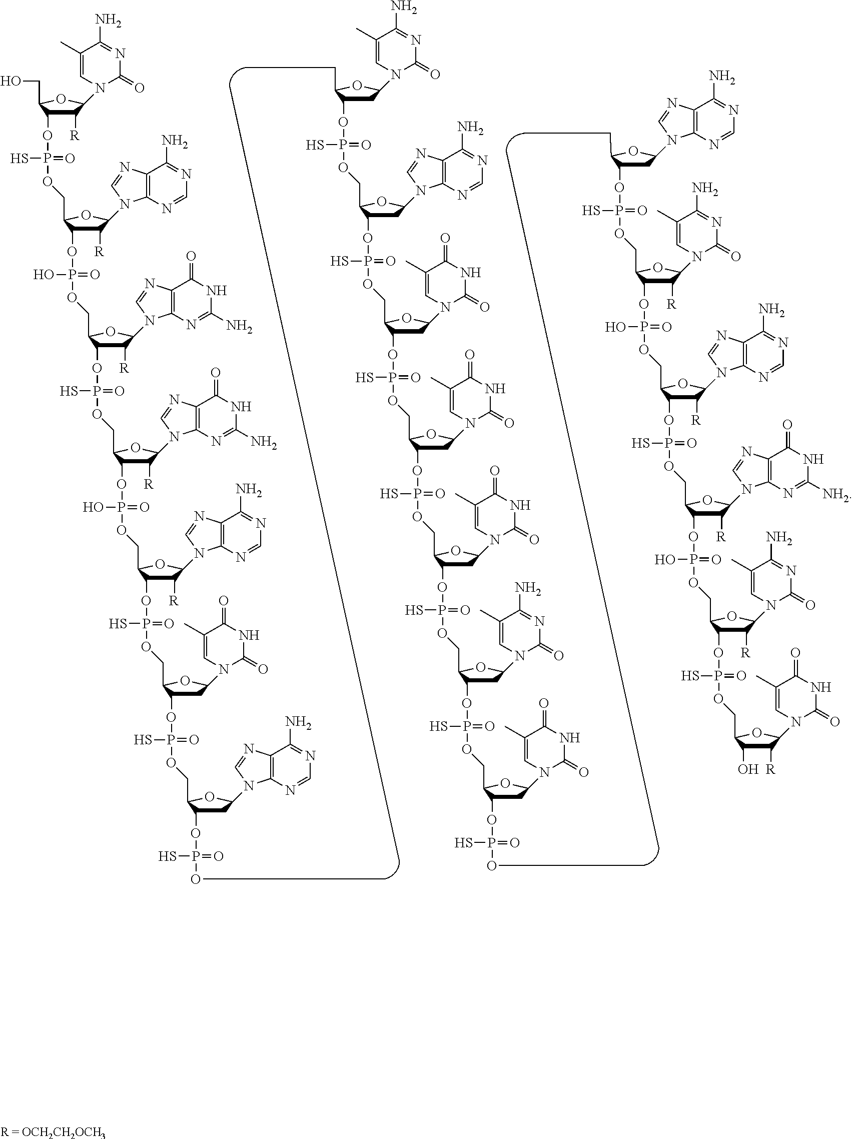

It is to be understood that in solution the antisense oligonucleotide may exist in free acid form, in a salt form, or a mixture thereof.

### Nonadecasodium Salt of ISIS 666853 (“Compound A”)

Compound A is a nonadecasodium salt of ISIS 666853, an antisense oligonucleotide inhibitor of SOD1 messenger ribonucleic acid (mRNA) that reduces the levels of SOD1 protein in subjects with SOD1 ALS. Reducing SOD1 mRNA and, subsequently, toxic SOD1 protein can offer therapeutic benefit for subjects with SOD1 ALS.

The structure of Compound A is provided below:

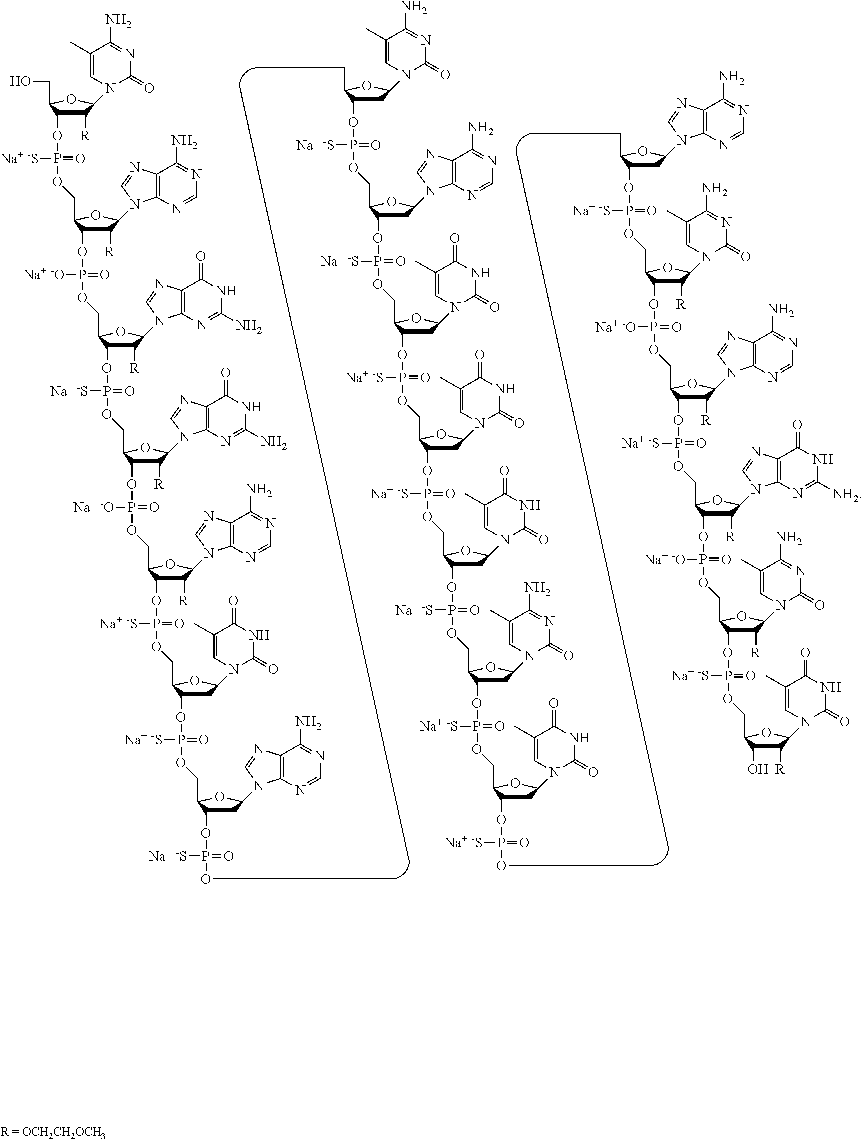

The molecular formula of Compound A is: C₂₃₀H₂₉₈N₇₂Na₁₉O₁₂₃P₁₉S₁₅, which has a molecular weight=7545.59 amu.

Compound A is complementary to a portion of the 3′ untranslated region (3′UTR) of the mRNA for human SOD1, binding by Watson Crick base pairing. The hybridization (binding) of Compound A to the cognate mRNA results in RNase-H1-mediated degradation of the mRNA for SOD1, and thus reduces the amount of SOD1 protein synthesis. RNase H is a ubiquitously expressed enzyme (nuclease) that recognizes a deoxyribonucleic acid-ribonucleic acid (DNA-RNA) heteroduplex and cleaves the RNA strand of the duplex. By binding to the 3′-UTR region of SOD1 mRNA, Compound A selectively targets RNase H1 to the SOD1 mRNA and promotes its cleavage, which leads to reduced expression of both wild-type and mutant variants of SOD1.

Compound A significantly increased the median survival in SOD1 G93A transgenic mice (Mantel-Cox, p<0.01). It also caused a dose-dependent protection of neuromuscular function, as measured by compound muscle action potential in SOD1 G93A transgenic mice.

### Conjugated Antisense Oligonucleotides

Antisense oligonucleotides of this disclosure may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes. Antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′stabilizing groups that can be used to cap one or both ends of an antisense oligonucleotide to impart nuclease stability include those disclosed in WO 03/004602.

### Compositions and Methods for Formulating Pharmaceutical Compositions

Antisense oligonucleotides or salts thereof of this disclosure may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense oligonucleotide, or salt thereof, targeted to a SOD1 nucleic acid can be used in pharmaceutical compositions by combining the antisense oligonucleotide, or salt thereof, with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense oligonucleotide, or salt thereof, targeted to a SOD1 nucleic acid and a pharmaceutically acceptable diluent.

An antisense oligonucleotide, or salt thereof, described herein may be formulated as a pharmaceutical composition for intrathecal administration to a subject.

Pharmaceutical compositions comprising antisense oligonucleotides of this disclosure encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligonucleotides and other bioequivalents. Suitable pharmaceutically acceptable salts include, but are not limited to, sodium and potassium salts.

### Methods of Treatment

The disclosure features methods of treating or preventing amyotrophic lateral sclerosis associated with a mutation in the human SOD1 gene in a human subject in need thereof. The method involves administering to the human subject by intrathecal administration a fixed dose of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. In certain instances, the fixed dose of the antisense oligonucleotide is about 100 mg or 100 mg. In other instances, the fixed dose of the antisense oligonucleotide is about 60 mg or 60 mg. In yet other instances, the fixed dose of the antisense oligonucleotide is about 40 mg or 40 mg. In some other instances, the fixed dose of the antisense oligonucleotide is about 20 mg or 20 mg. In certain instances, the fixed dose of the sodium salt of the antisense oligonucleotide is about 105.9 mg or 105.9 mg. In other instances, the fixed dose of the sodium salt of the antisense oligonucleotide is about 63.5 mg or 63.5 mg. In yet other instances, the fixed dose of the sodium salt of the antisense oligonucleotide is about 42.3 mg or 42.3 mg. In some other instances, the fixed dose of the sodium salt of the antisense oligonucleotide is about 21.2 mg or 21.2 mg.

Also provided are methods of reducing human SOD1 protein synthesis in a human subject having a mutation in the human SOD1 gene associated with amyotrophic lateral sclerosis. The method involves administering to the human subject by intrathecal administration a fixed dose of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. In certain instances, the fixed dose of the antisense oligonucleotide is about 100 mg or 100 mg. In other instances, the fixed dose of the antisense oligonucleotide is about 60 mg or 60 mg. In yet other instances, the fixed dose of the antisense oligonucleotide is about 40 mg or 40 mg. In some other instances, the fixed dose of the antisense oligonucleotide is about 20 mg or 20 mg.

Also provided are methods of reducing human SOD1 mRNA levels in a human subject having a mutation in the human SOD1 gene associated with amyotrophic lateral sclerosis. The method involves administering to the human subject by intrathecal administration a fixed dose of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine. In certain instances, the fixed dose of the antisense oligonucleotide is about 100 mg or 100 mg. In other instances, the fixed dose of the antisense oligonucleotide is about 60 mg or 60 mg. In yet other instances, the fixed dose of the antisense oligonucleotide is about 40 mg or 40 mg. In some other instances, the fixed dose of the antisense oligonucleotide is about 20 mg or 20 mg.

Also provided are methods of treating or preventing amyotrophic lateral sclerosis associated with a mutation in the SOD1 gene in a human subject in need thereof, wherein the method entails administering to the human subject by intrathecal administration a pharmaceutical composition comprising an antisense oligonucleotide or a salt thereof, wherein the antisense oligonucleotide has the following structure:

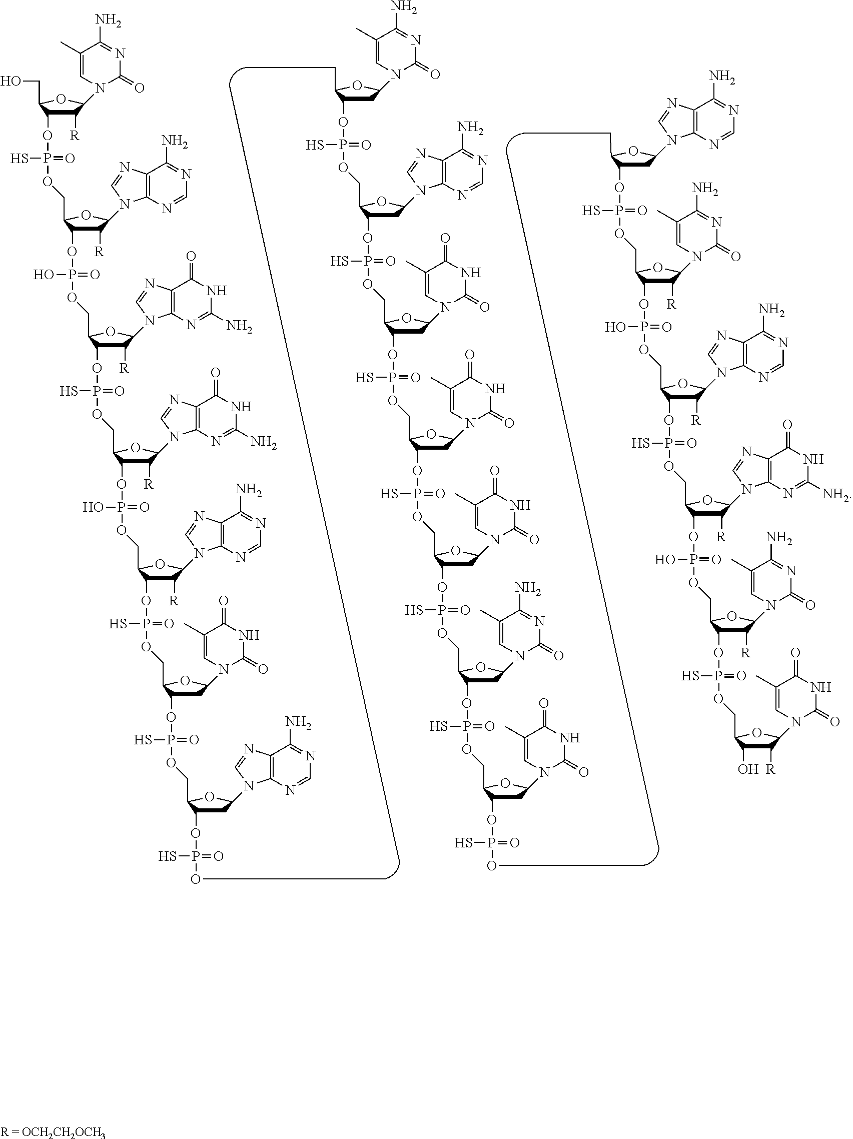

In certain instances, the antisense oligonucleotide or the salt thereof is administered at a dose equivalent to about 100 mg or 100 mg of the antisense oligonucleotide. In other instances, the antisense oligonucleotide or the salt thereof is administered at a dose equivalent to about 60 mg or 60 mg of the antisense oligonucleotide. In other instances, the antisense oligonucleotide or the salt thereof is administered at a dose equivalent to about 40 mg or 40 mg of the antisense oligonucleotide. In other instances, the antisense oligonucleotide or the salt thereof is administered at a dose equivalent to about 20 mg or 20 mg of the antisense oligonucleotide.

Also provided are methods of reducing human SOD1 protein synthesis in a human subject having a mutation in the human SOD1 gene associated with amyotrophic lateral sclerosis, wherein the method entails administering to the human subject by intrathecal administration a pharmaceutical composition comprising an antisense oligonucleotide or a salt thereof, wherein the antisense oligonucleotide has the following structure:

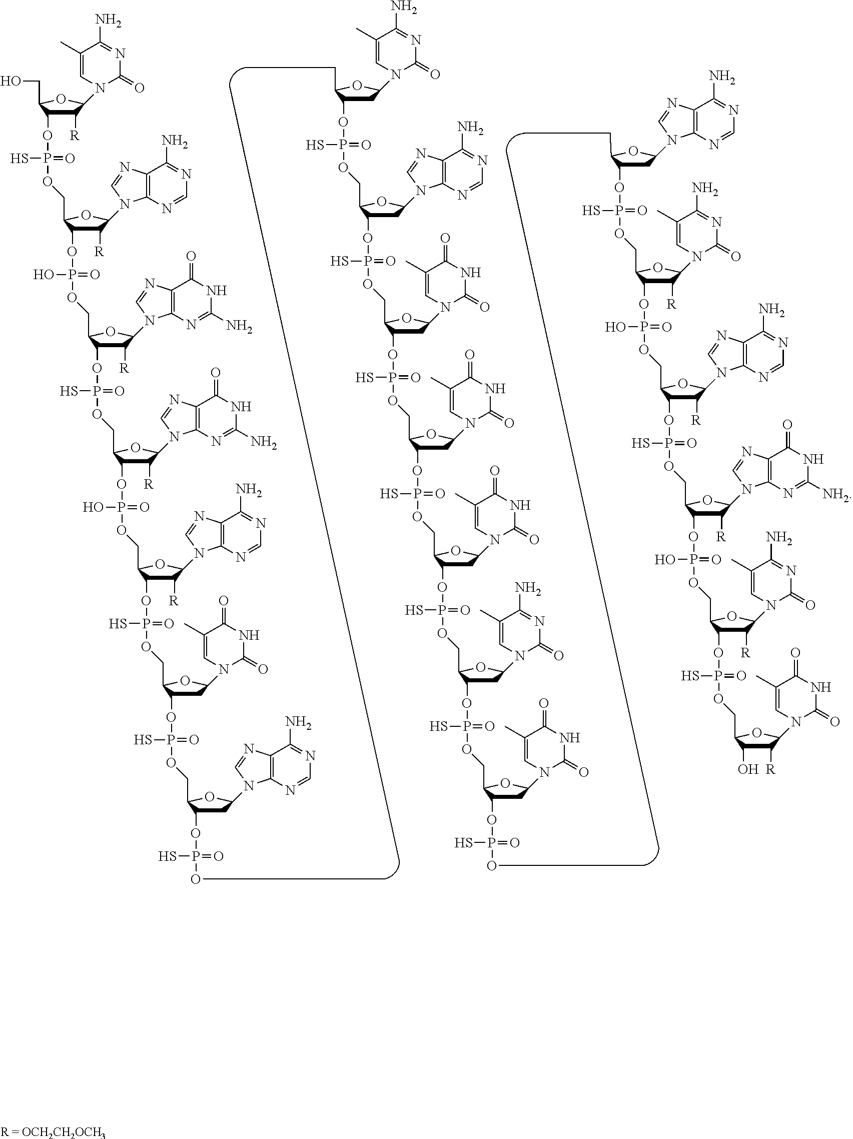

In certain instances, the antisense oligonucleotide or the salt thereof is administered at a dose equivalent to about 100 mg or 100 mg of the antisense oligonucleotide. In other instances, the antisense oligonucleotide or the salt thereof is administered at a dose equivalent to about 60 mg or 60 mg of the antisense oligonucleotide. In other instances, the antisense oligonucleotide or the salt thereof is administered at a dose equivalent to about 40 mg or 40 mg of the antisense oligonucleotide. In other instances, the antisense oligonucleotide or the salt thereof is administered at a dose equivalent to about 20 mg or 20 mg of the antisense oligonucleotide.

Also provided are methods of reducing human SOD1 mRNA levels in a human subject having a mutation in the human SOD1 gene associated with amyotrophic lateral sclerosis, wherein the method entails administering to the human subject by intrathecal administration a pharmaceutical composition comprising an antisense oligonucleotide or a salt thereof, wherein the antisense oligonucleotide has the following structure:

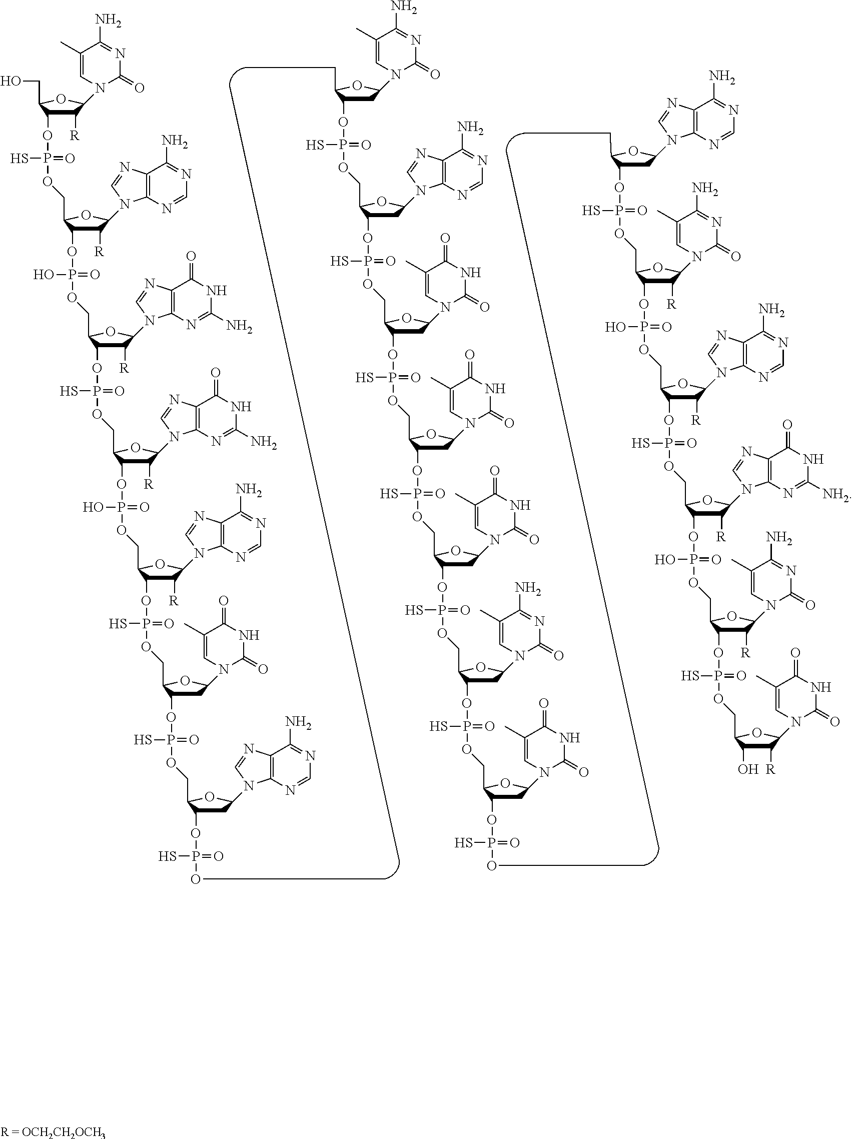

In certain instances, the antisense oligonucleotide or the salt thereof is administered at a dose equivalent to about 100 mg or 100 mg of the antisense oligonucleotide. In other instances, the antisense oligonucleotide or the salt thereof is administered at a dose equivalent to about 60 mg or 60 mg of the antisense oligonucleotide. In other instances, the antisense oligonucleotide or the salt thereof is administered at a dose equivalent to about 40 mg or 40 mg of the antisense oligonucleotide. In other instances, the antisense oligonucleotide or the salt thereof is administered at a dose equivalent to about 20 mg or 20 mg of the antisense oligonucleotide.

In some instances, an above-noted fixed dose of the antisense oligonucleotide, or salt thereof, is administered to the human subject once every week, once every two weeks, once every three weeks, or once every four weeks.

In some instances, the antisense oligonucleotide described herein is administered to the human subject as part of a pharmaceutical composition. In certain embodiments, the pharmaceutical composition is administered to the human subject in an amount sufficient to deliver a fixed dose of (i.e., the equivalent of) about 20 mg of the antisense oligonucleotide. In certain embodiments, the pharmaceutical composition is administered to the human subject in an amount sufficient to deliver a fixed dose of about 40 mg of the antisense oligonucleotide. In certain embodiments, the pharmaceutical composition is administered to the human subject in an amount sufficient to deliver a fixed dose of about 60 mg of the antisense oligonucleotide. In certain embodiments, the pharmaceutical composition is administered to the human subject in an amount sufficient to deliver a fixed dose of about 100 mg of the antisense oligonucleotide.

In certain embodiments, an above-noted fixed dose of the antisense oligonucleotide, or salt thereof, is administered as a loading dose(s). In some embodiments, an above-noted fixed dose of the antisense oligonucleotide is administered as a maintenance dose(s). In certain instances, the above-noted fixed dose of the antisense oligonucleotide is administered as a loading dose(s) and followed by a maintenance dose(s). The loading dose(s) can be administered, e.g., every week, every two weeks, every three weeks, or every four weeks. The maintenance dose(s) can be administered, e.g., every week, every two weeks, every three weeks, or every four weeks after the last loading dose. In some instances, the maintenance dose(s) is administered every month.

In certain embodiments, the human subject is administered three loading doses of the antisense oligonucleotide, or salt thereof, followed by at least one (e.g., 1, 2, 3, 4, 5, 6, 7, 8, 9, 10, 11, 12, or more) maintenance dose. In some instances, the three loading doses are administered two weeks apart. In some instances, the three loading doses are administered 14 days apart. In some instances, the maintenance dose/doses are administered beginning 4 weeks after the third loading dose. In some instances, the maintenance dose/doses are administered every month beginning after the third loading dose. In some instances, the maintenance dose/doses are administered every 28 days beginning after the third loading dose.

The mutation in SOD1 may be any mutation in the human SOD1 gene that is linked to ALS. In some instances, the mutation is a slow-progressing ALS disease mutation. In other instances, the mutation is a fast-progressing ALS disease mutation. In certain instances, the mutation in the human SOD1 gene is one or more of A4V, H46R, G93S, A4T, G141X, D133A, V148G, N139K, G85R, G93A, V14G, C6S, I113T, D49K, G37R, A89V, E100G, D90A, T137A, E100K, G41A, G41D, G41S, G13R, G72S, L8V, F20C, Q22L, H48R, T54R, S591, V87A, T88deltaTAD, A89T, V97M, S105deltaSL, V118L, D124G, L114F, D90A, G12R, or G147R. In one particular embodiment, the human subject has an A4V mutation in the human SOD1 gene. In another particular embodiment, the human subject has an H46R mutation in the human SOD1 gene. In yet another particular embodiment, the human subject has a G93S mutation in the human SOD1 gene.

In certain instances, the mutation in the SOD1 gene is identified by a genetic test.

In some instances, the methods described above involve identifying the mutation in the SOD1 gene by a genetic test.

In certain embodiments, administration of a therapeutically effective amount of an antisense oligonucleotide, or a salt thereof (e.g., Compound A), to a human subject is accompanied by monitoring of SOD1 levels in the human subject, to determine the human subject's response to administration of the antisense oligonucleotide, or salt thereof. A human subject's response to administration of the antisense oligonucleotide, or a salt thereof, may be used by a physician to determine the amount and duration of therapeutic intervention. In certain embodiments, the human SOD1 levels are monitored in CSF. In certain embodiments, the human SOD1 levels are monitored in plasma.

In certain embodiments, administration of an antisense oligonucleotide, or a salt thereof (e.g., Compound A), results in reduction of SOD1 protein expression. In certain embodiments, administration of an antisense oligonucleotide, or a salt thereof (e.g., Compound A), results in reduction of SOD1 protein expression by at least 10, 15, 20, 25, 30, 35, 40, 45, 50, 55, 60, 65, 70, 75, 80, 85, 90, 95 or 99%, or a range defined by any two of these values. In certain embodiments, the reduction of SOD1 protein expression is a reduction in the CSF. In certain embodiments, the reduction of SOD1 protein expression is a reduction in the plasma.

In certain embodiments, administration of an antisense oligonucleotide, or a salt thereof (e.g., Compound A), results in improved motor function and respiration in the human subject. In certain embodiments, administration of the antisense oligonucleotide, or salt thereof, improves motor function and respiration by at least 10, 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense oligonucleotide, or a salt thereof (e.g., Compound A), are used for the preparation of a medicament for treating a human subject suffering or susceptible to ALS (e.g., a human subject having a mutation in SOD1 linked to ALS).

### Delivery Devices

In certain embodiments, an antisense oligonucleotide, or a salt thereof (e.g., Compound A), is administered to the human subject with a syringe for intrathecal delivery. In another embodiment, an antisense oligonucleotide, or a salt thereof (e.g., Compound A), is administered to the human subject with a pump for intrathecal delivery. Thus, this disclosure also provides a pump or syringe comprising a sterile preparation of the antisense oligonucleotide, or a salt thereof (e.g., Compound A). The syringe or pump can be adapted for intrathecal administration of the antisense oligonucleotide, or a salt thereof. In some cases, the syringe or pump delivers a fixed dose(s) (e.g., about 20 mg or 20 mg, about 40 mg or 40 mg, about 60 mg or 60 mg, or about 100 mg or 100 mg) of the antisense oligonucleotide. The disclosure also provides a pump or syringe comprising a sterile preparation of a pharmaceutical composition comprising an antisense oligonucleotide, or a salt thereof (e.g., Compound A). The syringe or pump can be adapted for intrathecal administration of the pharmaceutical composition. In some cases, the syringe or pump delivers a fixed dose(s) (e.g., about 20 mg or 20 mg, about 40 mg or 40 mg, about 60 mg or 60 mg, or about 100 mg or 100 mg of the antisense oligonucleotide of the pharmaceutical composition. In a particular embodiment, the pump or syringe comprises a sterile preparation of an antisense oligonucleotide, or salt thereof, wherein the syringe or pump is adapted for intrathecal administration of an antisense oligonucleotide, or a salt thereof (e.g., Compound A), at a fixed dose of 20 mg, 40 mg, 60 mg, or 100 mg of the antisense oligonucleotide.

### Assays

### RNA Isolation

RNA analysis can be performed on total cellular RNA or poly(A)+ mRNA. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a SOD1 nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of SOD1 RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invitrogen, Inc. Eugene, Oreg.). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a SOD1 nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of SOD1 nucleic acids can be assessed by measuring SOD1 protein levels. Protein levels of SOD1 can be evaluated or quantitated in a variety of ways well known in the art, such as immunoprecipitation, Western blot analysis (immunoblotting), enzyme linked immunosorbent assay (ELISA), quantitative protein assays, protein activity assays (for example, caspase activity assays), immunohistochemistry, immunocytochemistry or fluorescence activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of human SOD1 are commercially available.

### Testing for SOD1 Mutations

One underlying genetic cause for ALS is a mutation(s) in the human SOD1 gene. Accordingly, identification of a subject suffering from or susceptible to ALS can be performed by genetic testing of the subject's SOD1 gene using assays known in the art, such as e.g., genetic sequencing. At least 180 mutations in human SOD1 are known in the art to be linked to ALS.

Analysis of a subject's susceptibility to ALS disease can also be performed by analyzing the family history of the subject for ALS. Analysis of the family history may include a three-generation pedigree documenting ALS, a review of medical records and autopsy studies of family members, and identification of an autosomal dominant pattern of SOD1 mutation.

The following example is not to be construed as limiting the scope of the invention in any way.

### EXAMPLES

### Example 1: Trial Design

Compound A is under investigation in ongoing clinical trials. The trials involve a randomized, placebo controlled, single ascending dose (SAD), and multiple ascending dose (MAD) study in SOD1 ALS patients. In the MAD portion, participants received 5 doses of study drug over approximately 3 months. Fifty participants were randomized (3:1 per cohort) to receive 20 mg, 40 mg, 60 mg, or 100 mg of Compound A, or placebo. Between 1 and 4 Compound A-treated participants per cohort had a documented SOD1 mutation that was adjudicated a priori to be fast-progressing (primarily A4V).

### Example 2: Safety Profile

In the trial, 66 of 70 patients (94%) experienced at least 1 adverse event (AE), most of which were graded as mild or moderate. The most common AEs occurring in ≥3 participants who received Compound A were headache (n=16), procedural pain (n=14), and postlumbar puncture syndrome (n=13). Seven patients experienced serious adverse events (SAEs), 3 of which were fatal events. No SAEs reported in the highest dose group (Compound A 100 mg). Six subjects had SAEs that were assessed as unrelated to Compound A and were felt to be related to ALS or comorbidities. All fatal events were assessed as unrelated. One patient had an SAE of CSF white blood cell increase and CSF protein increase assessed as related to Compound A. Those laboratory abnormalities resolved despite continued dosing with Compound A, and the patient completed the study. No serious adverse events were reported in the highest dose tested, 100 mg.

### Example 3: Pharmacokinetics and Pharmacodynamics

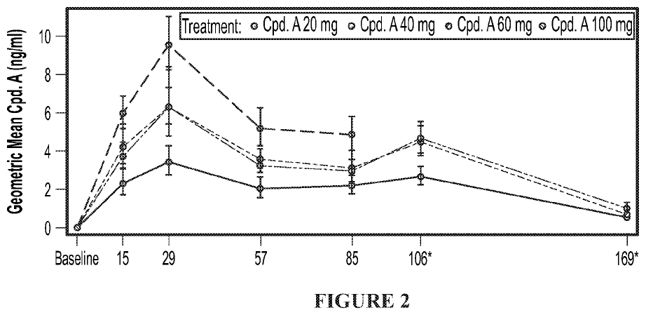

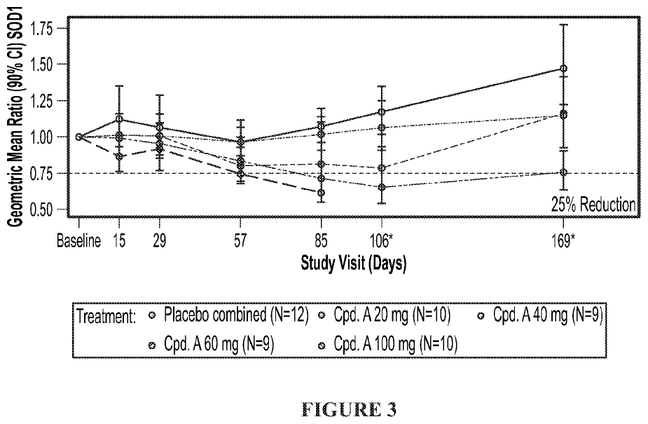

Dose dependent increases in Compound A plasma (not shown) and CSF concentrations were observed in the SAD (not shown) and MAD cohorts (FIG. 2). Plasma concentration of Compound A was dose proportional, measured at Days 1 and 85, while Compound A exposure in the CSF showed a less than dose-proportional response. Reductions from baseline in CSF SOD1 concentrations were observed at the 40 mg multiple dose level and above (i.e., 60 mg and 100 mg), which increased with dosage amount, with a maximum average reduction of 36% in the 100 mg multiple dose group (FIG. 3). Reductions from baseline in CSF SOD1 were observed in all participants in the 100 mg dose group. Maximum SOD1 reduction was observed at or immediately after the last dose, indicating that continuous dosing beyond 5 doses may yield further reductions. Modeling based on preclinical data suggest that Compound A 100 mg effectively reduces SOD1 levels in the spinal cord by >99% and approximately by 25-30% in the cortex.

### Example 4: Clinical Observations

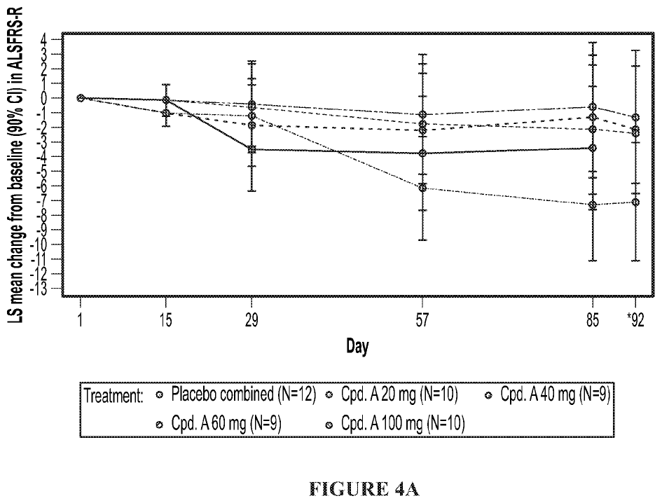

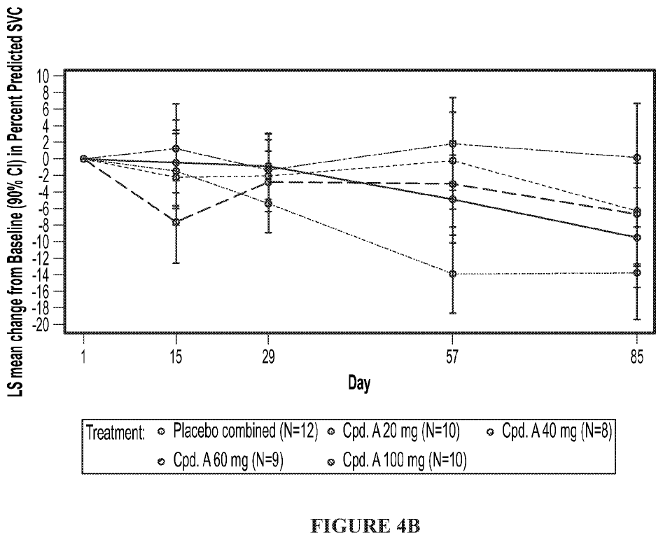

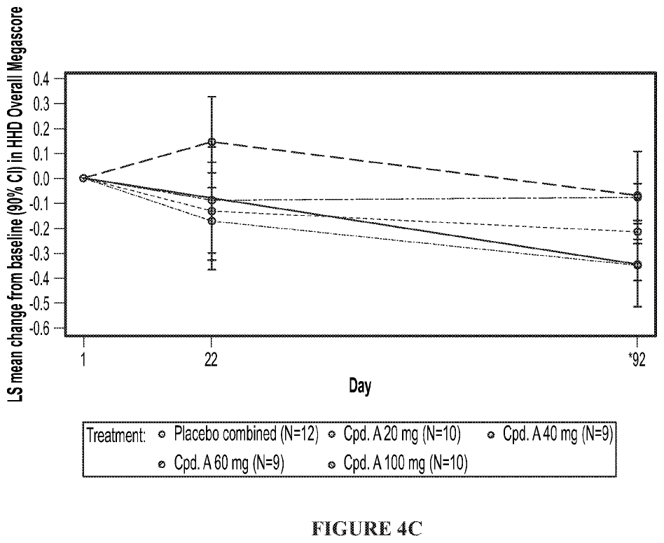

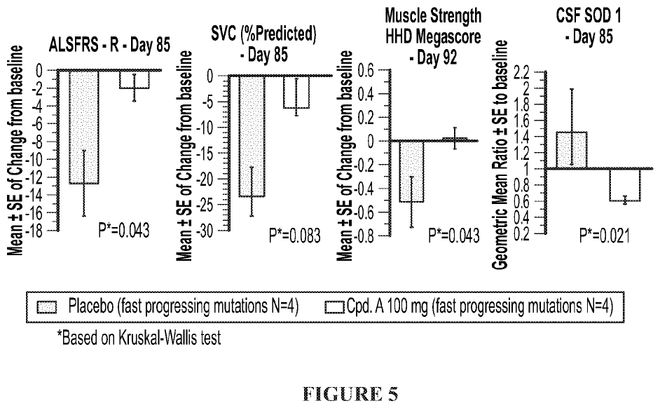

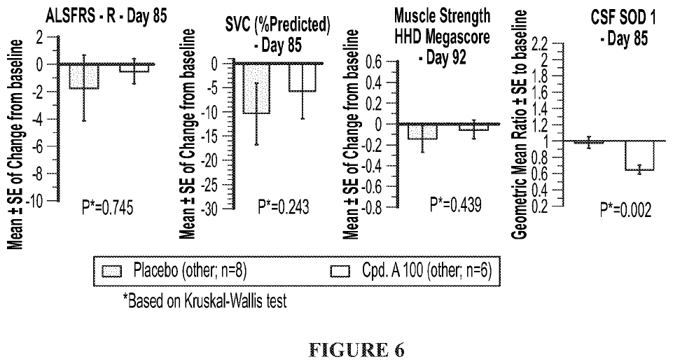

Efficacy was assessed at multiple time points on several scales including the Amyotrophic Lateral Sclerosis Functional Rating Scale-Revised (ALFSFRS-R), Slow Vital Capacity (SVC), and hand-held dynamometry (HHD) scales. In all cohorts, Compound A-treated groups had higher, i.e., better, scores compared to placebo-treated groups (FIGS. 4A to 4C). The mean change from baseline to Day 85 in ALSFRS-R scores for Compound A-treated participants at the 100 mg dose group (N=10) was −1.1 versus −5.6 for placebo treated participants (N=12); a difference of 4.4 points. In participants with SOD1 mutations that are known to be rapidly progressive, e.g., A4V, a marked difference between Compound A-treated participants at the 100 mg dose group and placebo treated participants was observed. In these fast progressing participants, the mean difference in change of ALSFRS-R from baseline to Day 85 approached 10 points (FIG. 5). The treatment effect appears to be consistent across multiple clinical scales and CSF SOD1 reduction in both fast-progressing (FIG. 5) and non-fast progressing participants (FIG. 6). As a frame of reference, the ALSFRS-R difference at 6 months was 2.5 points in the edaravone pivotal trial in ALS.

Taken together, the trial has demonstrated that Compound A is a safe and effective treatment for subjects with SOD1 ALS. This is reinforced most strongly by the outcomes in Compound A-treated subjects with fast progressing mutation subtypes (primarily A4V), particularly in the 100 mg dose group, for whom rapid decline would have been expected in the absence of an efficacious treatment. As noted above, efficacy was assessed at multiple time points on several scales including the Amyotrophic Lateral Sclerosis Functional Rating Scale-Revised (ALSFRS-R), slow vital capacity (SVC), and hand-held dynamometry (HHD). The results show a much smaller decline for each of the 3 clinical function endpoints in the Compound A 100 mg dose group compared to the placebo group.

### Example 5: CSF Phosphorylated Neurofilament Heavy Chain (pNFH) Levels

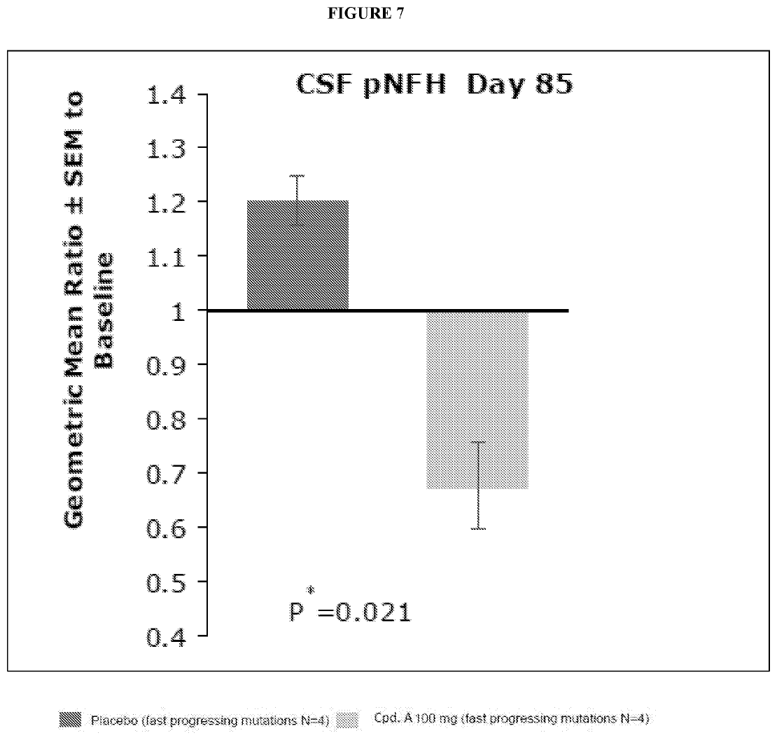

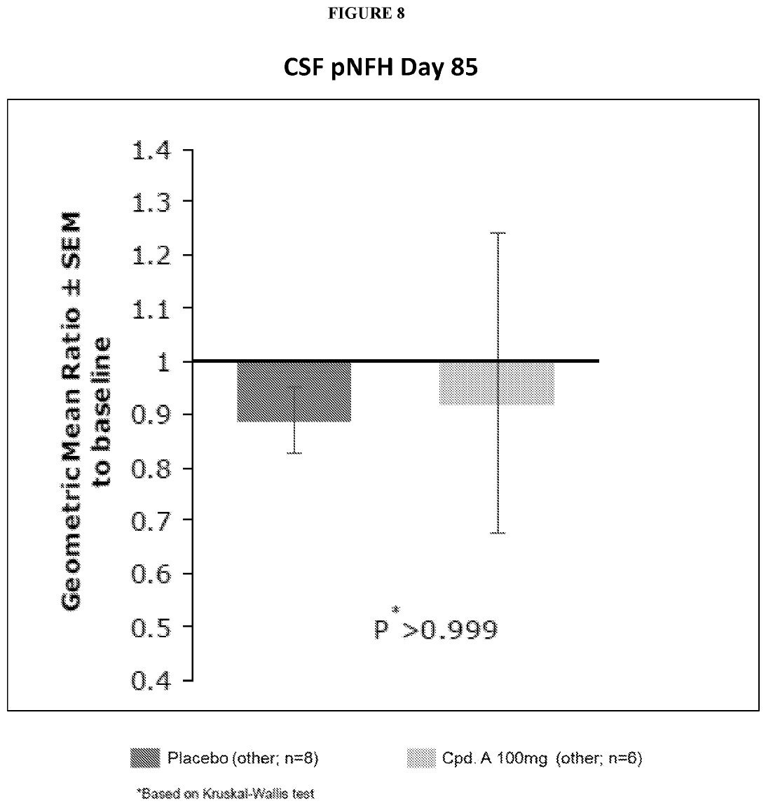

In patients with fast-progressing SOD1 mutations, treatment with Compound A resulted in a reduction in CSF pNFH levels and a slowing of clinical decline compared to placebo. A greater difference in pNFH levels at day 85 between the Compound A 100 mg and placebo groups was observed in patients with fast-progressing SOD1 mutations (FIG. 7), compared to patients with other SOD1 mutations (FIG. 8).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition in an amount sufficient to deliver a fixed dose of about 100 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

2. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition in an amount sufficient to deliver a fixed dose of about 60 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

3. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition in an amount sufficient to deliver a fixed dose of about 40 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

4. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition in an amount sufficient to deliver a fixed dose of about 20 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

5. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition in an amount sufficient to deliver a fixed dose of about 100 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

6. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition in an amount sufficient to deliver a fixed dose of about 60 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

7. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition in an amount sufficient to deliver a fixed dose of about 40 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

8. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition in an amount sufficient to deliver a fixed dose of about 20 mg of an antisense oligonucleotide, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

9. The method of any one of claims 1 to 8, wherein the mutation in the SOD1 gene is A4V.

10. The method of any one of claims 1 to 8, wherein the mutation in the SOD1 gene is A4V, H46R, G93S, A4T, G141X, D133A, V148G, N139K, G85R, G93A, V14G, C6S, I113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, or G147R.

11. The method of any one of claims 1 to 10, wherein the mutation in the SOD1 gene is identified by a genetic test.

12. The method of any one of claims 1 to 10, comprising identifying the mutation in the SOD1 gene by a genetic test.

13. The method of any one of claims 1 to 12, wherein the pharmaceutical composition is administered to the human subject at least 5 times over the course of four months.

14. The method of any one of claims 1 to 13, wherein the human subject is administered loading doses of the pharmaceutical composition followed by maintenance doses of the pharmaceutical composition.

15. The method of claim 14, wherein the human subject is administered three loading doses, and wherein the loading doses are administered two weeks apart.

16. The method of claim 14, wherein the maintenance doses are administered every 4 weeks beginning 4 weeks after the third loading dose.

17. The method of claim 14, wherein the loading doses and maintenance doses of the pharmaceutical composition are administered to the human subject as follows:
(i) a first loading dose of the pharmaceutical composition;
(ii) a second loading dose of the pharmaceutical composition administered 14 days after the first loading dose;
(iii) a third loading dose of the pharmaceutical composition administered 28 days after the first loading dose; and
(iv) a first maintenance dose of the pharmaceutical composition administered 28 days or 1 month after the third loading dose.

18. The method of claim 14, wherein the loading doses and maintenance doses of the pharmaceutical composition are administered to the human subject as follows:
(i) a first loading dose in an amount sufficient to deliver a fixed dose of about 100 mg of the antisense oligonucleotide;
(ii) a second loading dose in an amount sufficient to deliver a fixed dose of about 100 mg of the antisense oligonucleotide, wherein the second loading dose is administered 14 days after the first loading dose;
(iii) a third loading dose in an amount sufficient to deliver a fixed dose of about 100 mg of the antisense oligonucleotide, wherein the third loading dose is administered 28 days after the first loading dose; and
(iv) a first maintenance dose in an amount sufficient to deliver a fixed dose of about 100 mg of the antisense oligonucleotide, wherein the first maintenance dose is administered 28 days after the third loading dose.

19. The method of claim 14, wherein the loading doses and maintenance doses of the pharmaceutical composition are administered to the human subject as follows:
(i) a first loading dose in an amount sufficient to deliver a fixed dose of about 100 mg of the antisense oligonucleotide;
(ii) a second loading dose in an amount sufficient to deliver a fixed dose of about 100 mg of the antisense oligonucleotide, wherein the second loading dose is administered 14 days after the first loading dose;
(iii) a third loading dose in an amount sufficient to deliver a fixed dose of about 100 mg of the antisense oligonucleotide, wherein the third loading dose is administered 28 days after the first loading dose; and
(iv) a first maintenance dose in an amount sufficient to deliver a fixed dose of about 100 mg of the antisense oligonucleotide, wherein the first maintenance dose is administered 1 month after the third loading dose.

20. A syringe or pump comprising a sterile preparation of an antisense oligonucleotide, wherein the syringe or pump is adapted for intrathecal administration of the antisense oligonucleotide at a fixed dose of about 20 mg, about 40 mg, about 60 mg, or about 100 mg, wherein the nucleobase sequence of the antisense oligonucleotide consists of CAGGATACATTTCTACAGCT (SEQ ID NO:1), wherein each of nucleosides 1-5 and 16-20 are 2′-O-methoxyethylribose modified nucleosides, and each of nucleosides 6-15 are 2′-deoxynucleosides, wherein the internucleoside linkages between nucleosides 2 to 3, 4 to 5, 16 to 17, and 18 to 19 are phosphodiester linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 17 to 18, and 19 to 20 are phosphorothioate linkages, and wherein each cytosine is a 5-methylcytosine.

21. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition comprising an antisense oligonucleotide or a salt thereof, wherein the antisense oligonucleotide has the following structure: 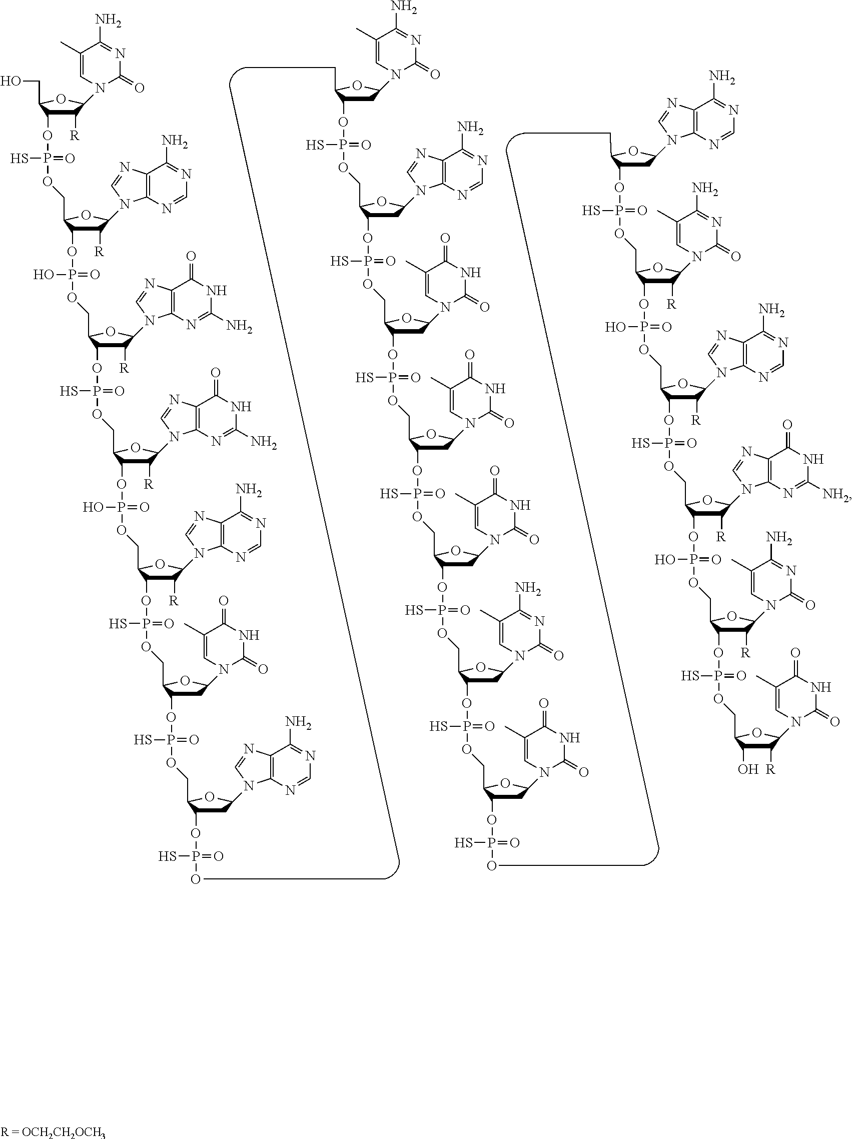
and wherein the antisense oligonucleotide or the salt thereof is administered at a dose equivalent to about 100 mg of the antisense oligonucleotide.

22. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition comprising an antisense oligonucleotide or a salt thereof, wherein the antisense oligonucleotide has the following structure: 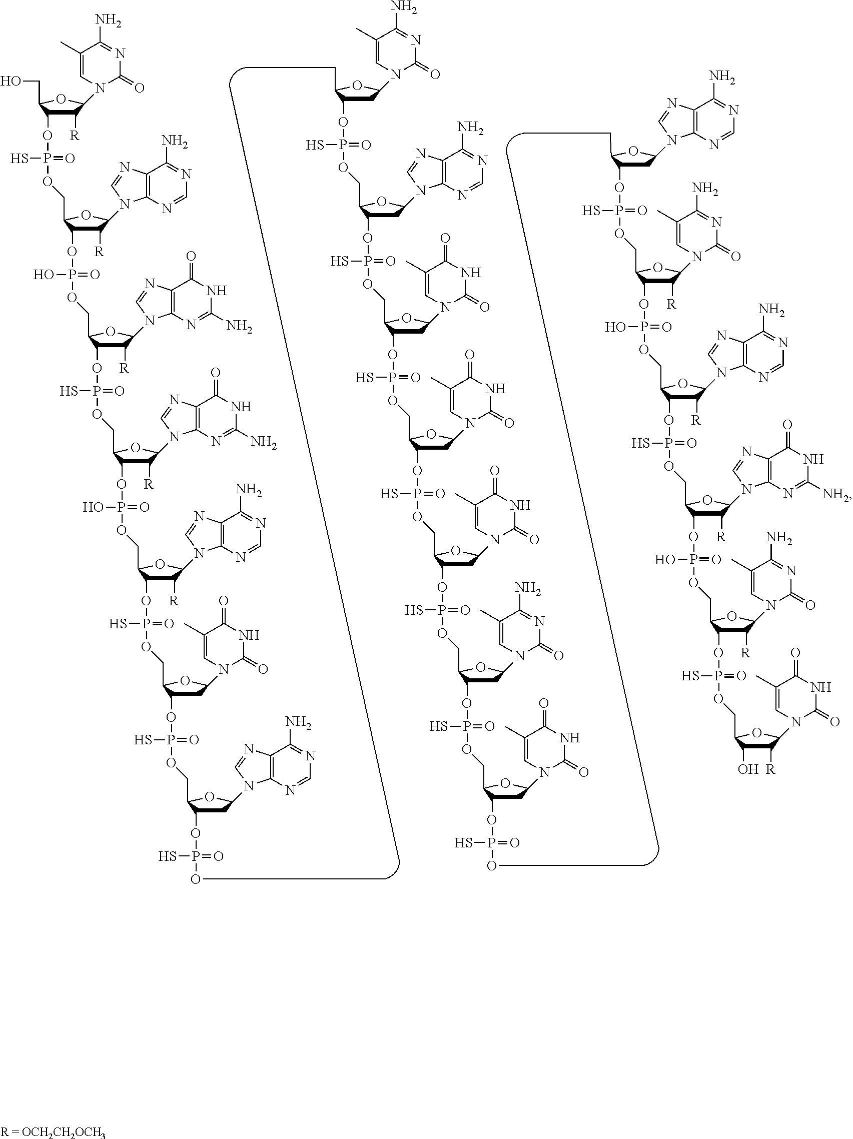
and wherein the antisense oligonucleotide or the salt thereof is administered at a dose equivalent to about 60 mg of the antisense oligonucleotide.

23. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition comprising an antisense oligonucleotide or a salt thereof, wherein the antisense oligonucleotide has the following structure: 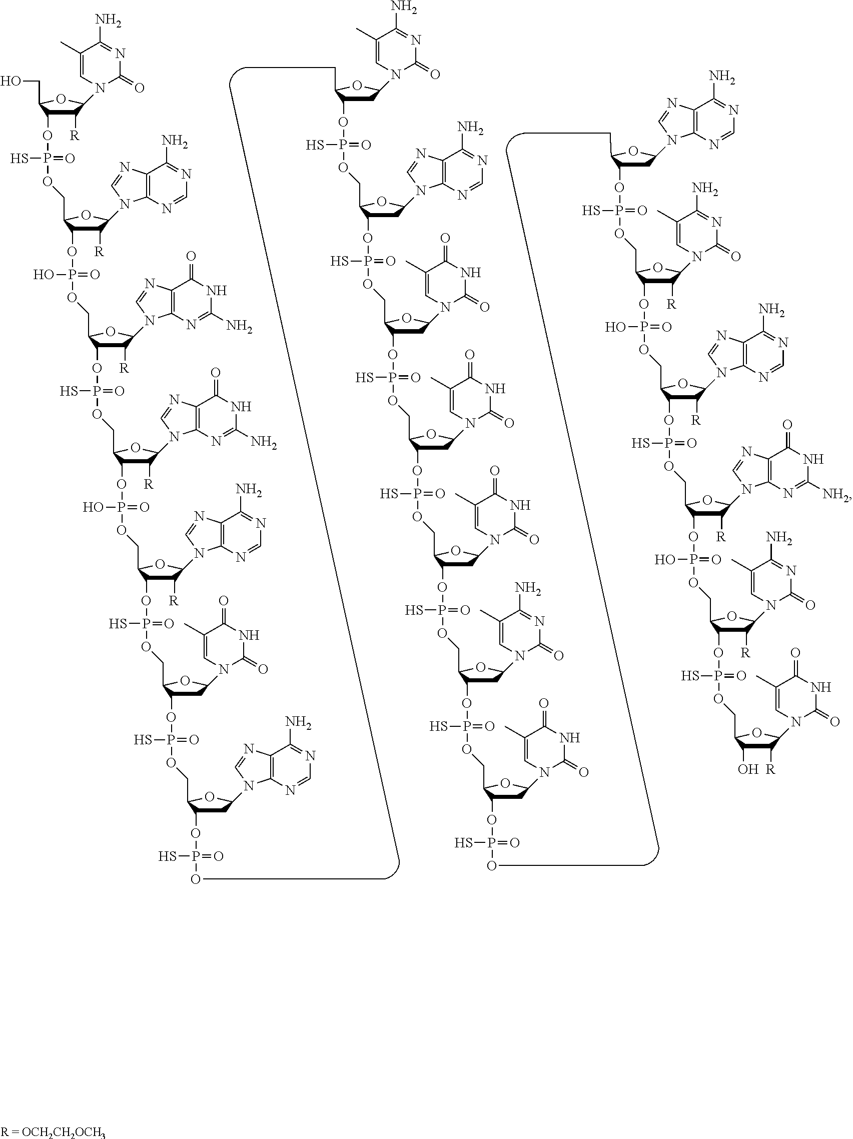
and wherein the antisense oligonucleotide or the salt thereof is administered at a dose equivalent to about 40 mg of the antisense oligonucleotide.

24. A method of treating or preventing amyotrophic lateral sclerosis associated with a mutation in the superoxide dismutase 1 (SOD1) gene in a human subject in need thereof, the method comprising administering to the human subject by intrathecal administration a pharmaceutical composition comprising an antisense oligonucleotide or a salt thereof, wherein the antisense oligonucleotide has the following structure: 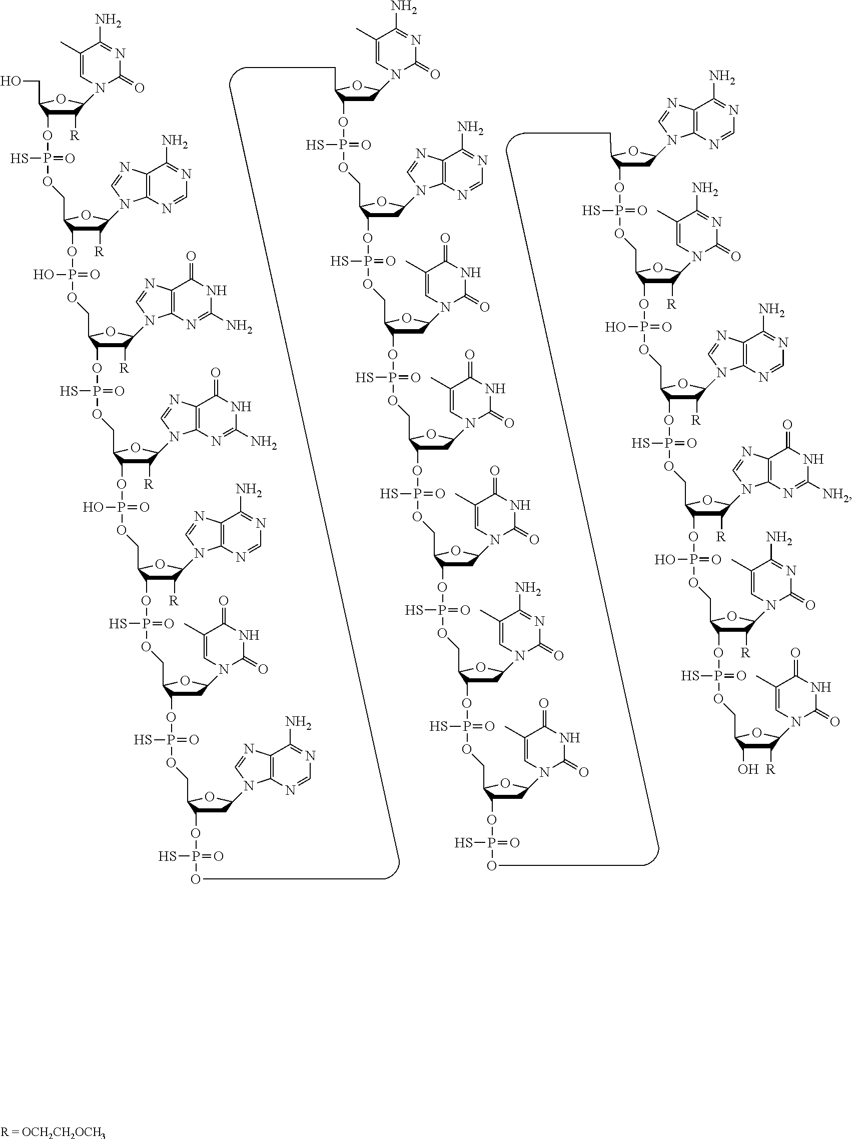
and wherein the antisense oligonucleotide or the salt thereof is administered at a dose equivalent to about 20 mg of the antisense oligonucleotide.

25. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition comprising an antisense oligonucleotide or a salt thereof, wherein the antisense oligonucleotide has the following structure: 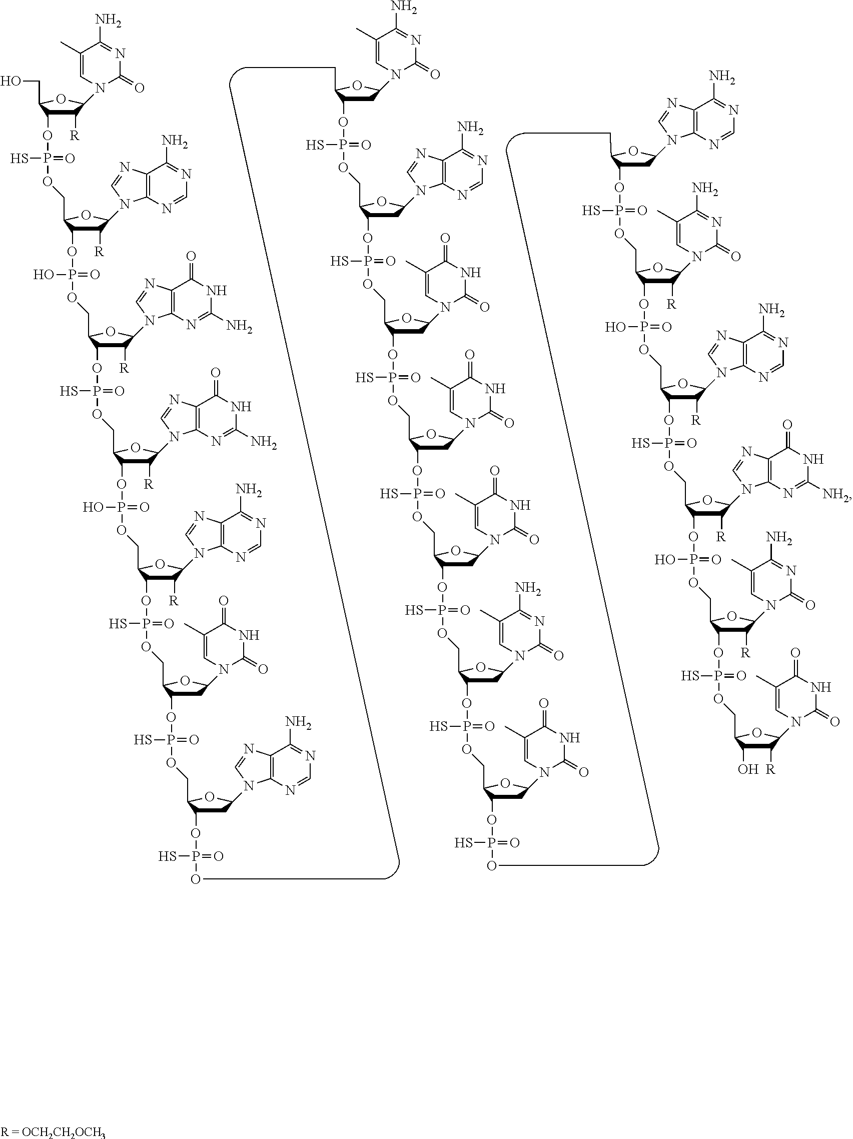
and wherein the antisense oligonucleotide or the salt thereof is administered at a dose equivalent to about 100 mg of the antisense oligonucleotide.

26. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition comprising an antisense oligonucleotide or a salt thereof, wherein the antisense oligonucleotide has the following structure: 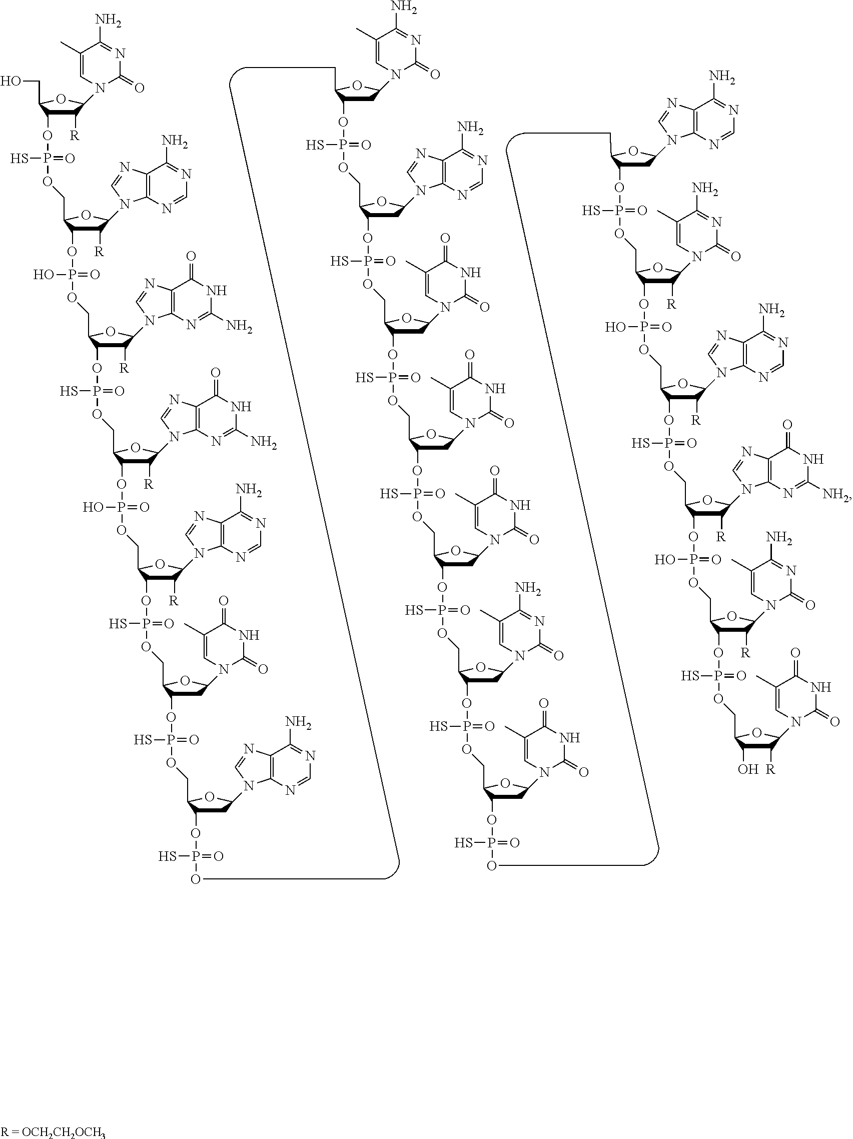
and wherein the antisense oligonucleotide or the salt thereof is administered at a dose equivalent to about 60 mg of the antisense oligonucleotide.

27. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition comprising an antisense oligonucleotide or a salt thereof, wherein the antisense oligonucleotide has the following structure: 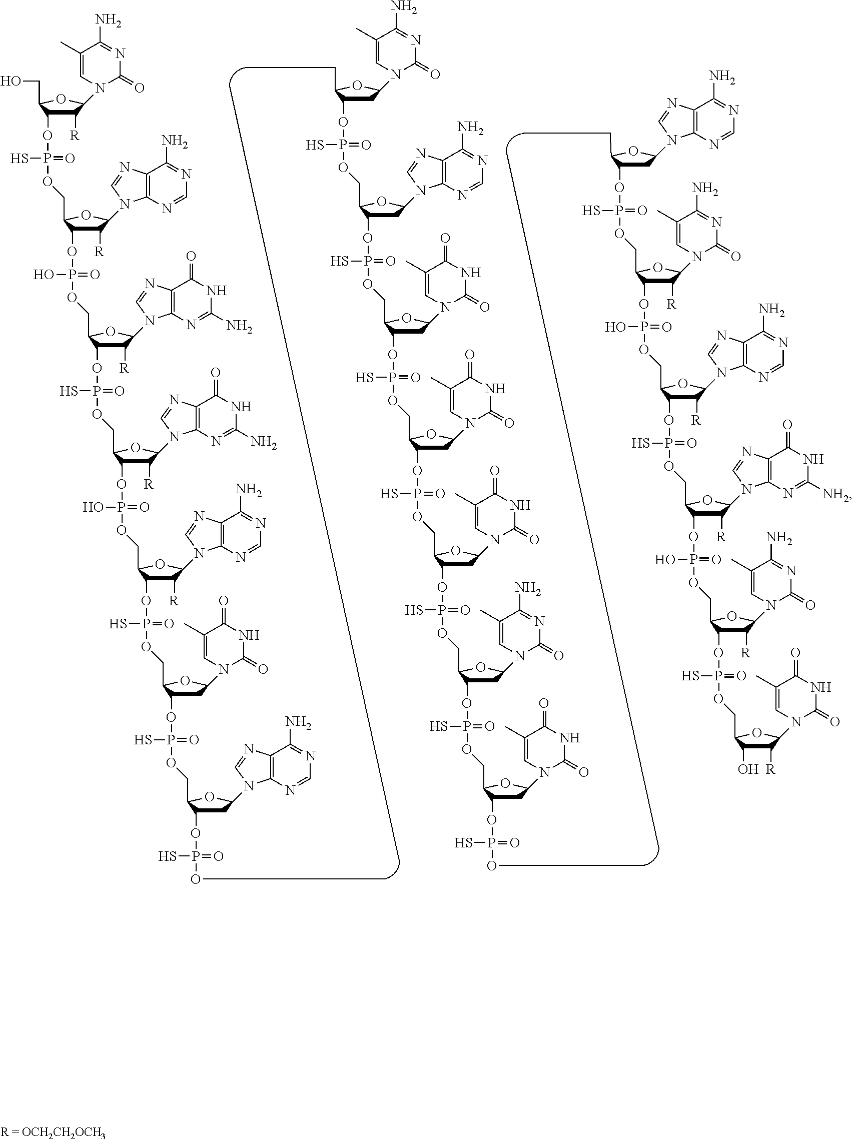
and wherein the antisense oligonucleotide or the salt thereof is administered at a dose equivalent to about 40 mg of the antisense oligonucleotide.

28. A method of reducing superoxide dismutase 1 (SOD1) protein synthesis in a human subject having a mutation in the SOD1 gene associated with amyotrophic lateral sclerosis, the method comprising administering to the human subject by intrathecal administration a pharmaceutical composition comprising an antisense oligonucleotide or a salt thereof, wherein the antisense oligonucleotide has the following structure: 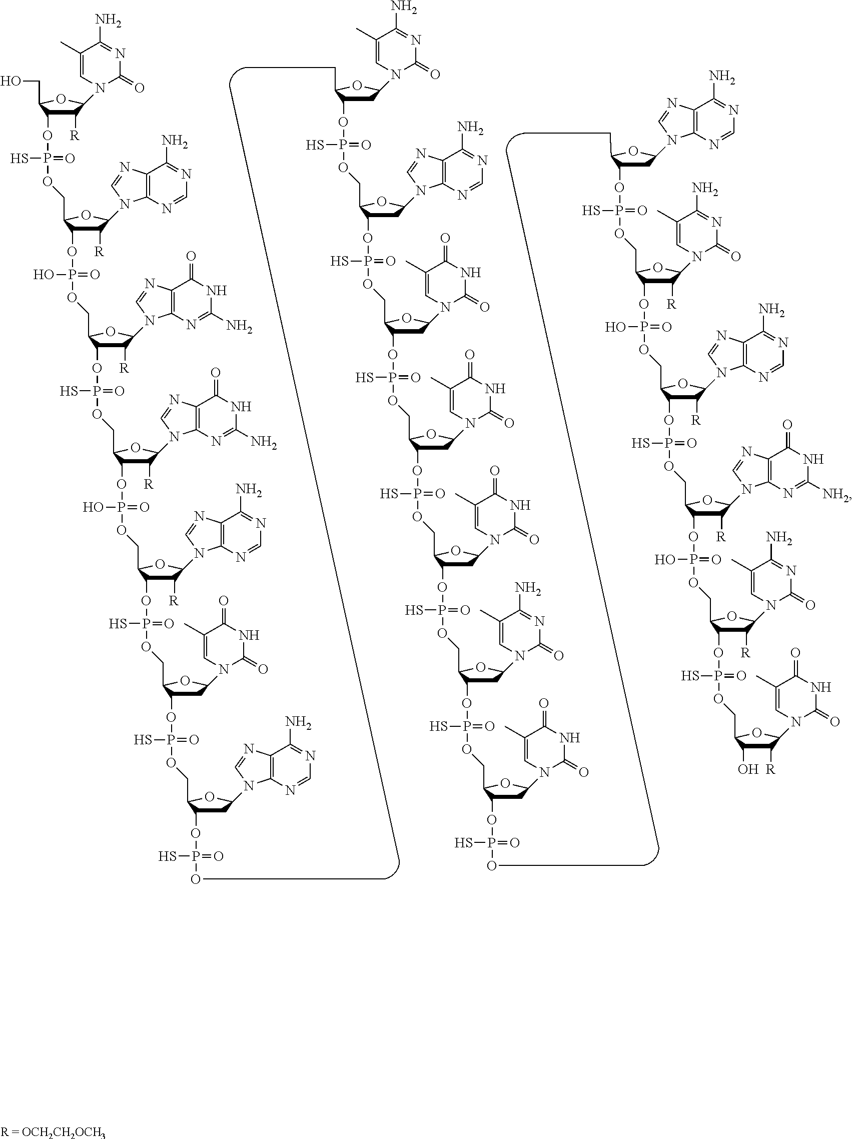
and wherein the antisense oligonucleotide or the salt thereof is administered at a dose equivalent to about 20 mg of the antisense oligonucleotide.

29. The method of any one of claims 21 to 28, wherein the mutation in the SOD1 gene is A4V.

30. The method of any one of claims 21 to 28, wherein the mutation in the SOD1 gene is A4V, H46R, G93S, A4T, G141X, D133A, V148G, N139K, G85R, G93A, V14G, C6S, I113T, D49K, G37R, A89V, E100G, D90A, T137A, E100K, G41A, G41D, G41S, G13R, G72S, L8V, F20C, Q22L, H48R, T54R, 5591, V87A, T88deltaTAD, A89T, V97M, S105deltaSL, V118L, D124G, L114F, D90A, G12R, or G147R.

31. The method of any one of claims 21 to 30, wherein the mutation in the SOD1 gene is identified by a genetic test.

32. The method of any one of claims 21 to 30, comprising identifying the mutation in the SOD1 gene by a genetic test.

33. The method of any one of claims 21 to 32, wherein the human subject is administered a salt of the antisense oligonucleotide.

34. The method of claim 33, wherein the salt is a sodium salt.

35. The method of claim 33, wherein the salt of the antisense oligonucleotide has the following structure:

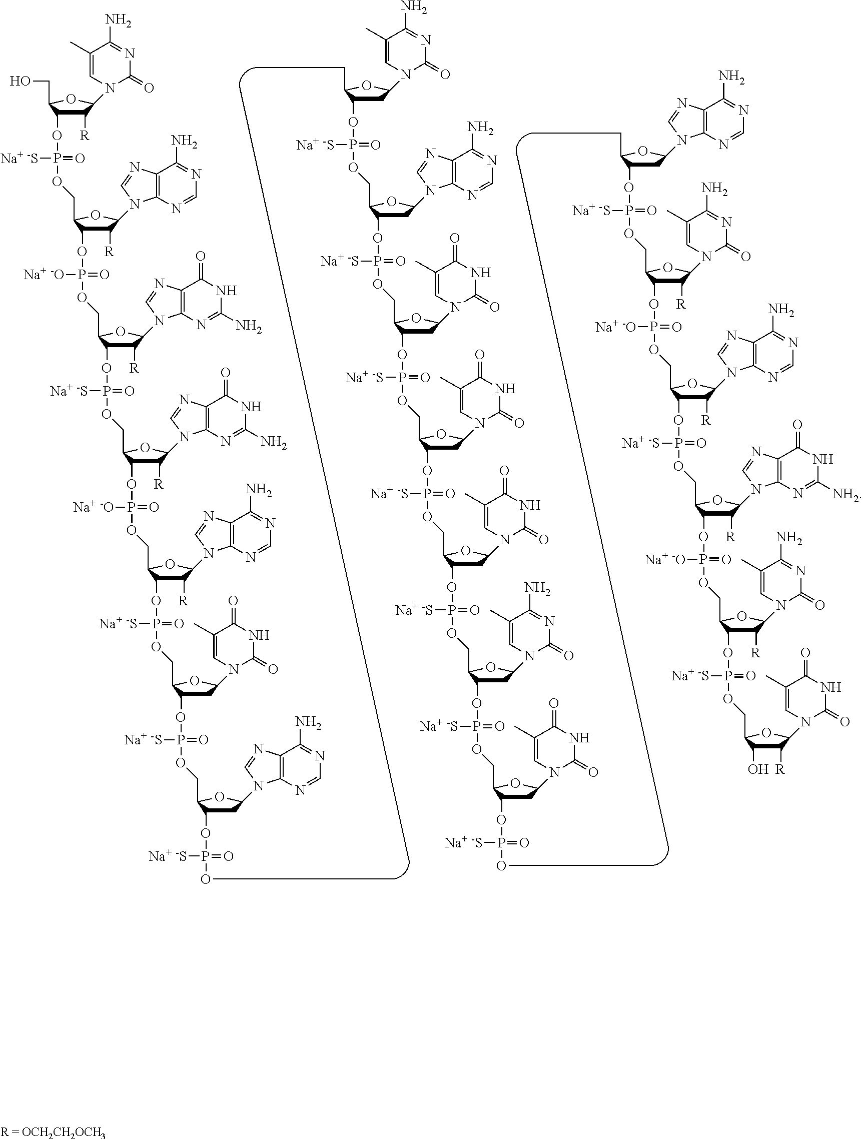

36. The method of any one of claims 21 to 35, wherein the human subject is administered loading doses of the pharmaceutical composition followed by maintenance doses of the pharmaceutical composition.

37. The method of claim 36, wherein the human subject is administered three loading doses, and wherein the loading doses are administered two weeks apart.

38. The method of claim 36, wherein the maintenance doses are administered every 4 weeks beginning 4 weeks after the third loading dose.

39. The method of claim 36, wherein the loading doses and maintenance doses of the pharmaceutical composition are administered to the human subject as follows:
(i) a first loading dose of the pharmaceutical composition;
(ii) a second loading dose of the pharmaceutical composition administered 14 days after the first loading dose;
(iii) a third loading dose of the pharmaceutical composition administered 28 days after the first loading dose; and
(iv) a first maintenance dose of the pharmaceutical composition administered 28 days or 1 month after the third loading dose.

40. The method of claim 36, wherein the loading doses and maintenance doses of the pharmaceutical composition are administered to the human subject as follows:
(i) a first loading dose equivalent to about 100 mg of the antisense oligonucleotide;
(ii) a second loading dose equivalent to about 100 mg of the antisense oligonucleotide, wherein the second loading dose is administered 14 days after the first loading dose;
(iii) a third loading dose equivalent to about 100 mg of the antisense oligonucleotide, wherein the third loading dose is administered 28 days after the first loading dose; and
(iv) a first maintenance dose equivalent to about 100 mg of the antisense oligonucleotide, wherein the first maintenance dose is administered 28 days after the third loading dose.

41. The method of claim 36, wherein the loading doses and maintenance doses of the pharmaceutical composition are administered to the human subject as follows:
(i) a first loading dose equivalent to about 100 mg of the antisense oligonucleotide;
(ii) a second loading dose equivalent to about 100 mg of the antisense oligonucleotide, wherein the second loading dose is administered 14 days after the first loading dose;
(iii) a third loading dose equivalent to about 100 mg of the antisense oligonucleotide, wherein the third loading dose is administered 28 days after the first loading dose; and
(iv) a first maintenance dose equivalent to about 100 mg of the antisense oligonucleotide, wherein the first maintenance dose is administered 1 month after the third loading dose.
